(19) United States
(12) Patent Application Publication
(10) Pub. No.: US 2021/0311051 A1
(43) Pub. Date: Oct. 7, 2021

(54) SYSTEMS AND METHODS FOR DETECTING AND QUANTIFYING ANALYTES

(71) Applicant: NONIGENEX, INC, La Jolla, CA (US)

(72) Inventors: Jerome P. LAPOINTE, Santa Cruz, CA (US); Andrew SENYEI, La Jolla, CA (US)

(21) Appl. No.: 16/868,464

(22) Filed: May 6, 2020

Related U.S. Application Data
(60) Provisional application No. 63/005,168, filed on Apr. 3, 2020.

(51) Int. Cl. G01N 33/569; C07K 14/005; C07K 16/10; G01N 33/68; A61K 39/215; G01N 33/543

(52) U.S. Cl. CPC G01N 33/56983; C07K 14/005; C07K 16/1018; G01N 33/6854; G01N 2800/50; G01N 33/54366; G01N 2333/165; G01N 2800/52; A61K 39/215

(57) ABSTRACT

Described herein are systems and methods for detecting and quantifying analytes. Also described herein are systems and methods for detecting and quantifying analytes stemmed from infections.

### CROSS REFERENCE

This application claims the benefit of U.S. Provisional Application Ser. No. 63/005,168 filed on Apr. 3, 2020, the entirety of which is hereby incorporated by reference herein.

### SEQUENCE LISTING

The instant application contains a Sequence Listing which has been submitted electronically in ASCII format and is hereby incorporated by reference in its entirety. Said ASCII copy, created on May 6, 2020, is named 58552-701_201_SL.txt and is 20,750 bytes in size.

### BACKGROUND

Pathogenic infections on a cellular level are mediated by pathogens binding to receptors expressed on the surface of a target cell. For example, the spike glycoprotein of a coronavirus binds to the angiotensin-converting enzyme 2 (ACE2) receptor, and binding between the receptor-binding domain (RBD) of the spike protein and ACE2 precedes entry of the coronavirus into the cell. Individuals who are exposed to a new pathogen (e.g., coronavirus) may develop neutralizing antibodies against the pathogen to block pathogen infection. This adaptive immune response significantly reduces incidences of a second infection by the same pathogen.

### SUMMARY

Provided herein are portable, point of need, testing devices that detect one or more neutralizing antibodies in a biological sample from a subject that functionally block pathogen binding to its cognate receptor. Methods of using the devices described herein, comprise assaying a biological sample from a subject with the testing device and detecting a complex between the neutralizing antibodies and a detectable peptide-conjugate derived from the pathogen. In some embodiments, methods comprise detecting with the naked eye. Also provided are systems comprising the testing device and an imaging device, such as a smartphone. In some embodiments, detecting comprises capturing an image of a detection zone of the testing device, analyzing the data from the image, and providing a result to the subject. In some embodiments, the pathogen is severe acute respiratory syndrome coronavirus 2 (SARS-CoV-2).

Aspects disclosed herein comprise systems comprising: (a) one or more capture molecules derived from an angiotensin-converting enzyme 2 (ACE2) receptor; and (b) a peptide-conjugate comprising: (i) a peptide derived from a spike glycoprotein of a coronavirus; and (ii) a detectable moiety. In some embodiments, the system further comprises (a) a surface; and (b) an imaging device configured to capture an image of a complex between the peptide-conjugate and the one or more capture molecules on the surface when the complex is coupled to the surface. In some embodiments, the system further comprises an imaging device configured to capture an image of a complex between the peptide-conjugate and the one or more capture molecules. In some embodiments, the system further comprises a container comprising (a) and (b), wherein the container is portable.

In some embodiments, the system is a point of need system. In some embodiments, the point of need is a point of care system. In some embodiments, the surface comprises a material selected from the group consisting of a metal, a plastic, glass, and a nitrocellulose membrane. In some embodiments, the surface is a passivated surface. In some embodiments, the passivated surface comprises a polymer layer comprising a molecule selected from the group consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran. In some embodiments, the complex is coupled to the surface. In some embodiments, the complex is coupled to the surface by a covalent bond, a linker, or a combination thereof. In some embodiments, the linker is a chemical linker, a peptide linker, or a combination thereof. In some embodiments, the one or more capture molecules is coupled to the surface. In some embodiments, the one or more capture molecules is coupled to the surface by a covalent bond, a linker, or a combination thereof. In some embodiments, the linker is a chemical linker, a peptide linker, or a combination thereof. In some embodiments, the one or more capture molecules is a fusion polypeptide. In some embodiments, the fusion polypeptide comprises at least a portion of a fragment crystallizable region (Fc) region of a monoclonal antibody. In some embodiments, the one or more capture molecules is bound by an antibody that is coupled to the surface. In some embodiments, the peptide-conjugate comprises a nanoparticle, a fluorescent dye, an enzymatic label, or a colorimetric label, or a combination thereof. In some embodiments, the nanoparticle comprises a material selected from the group consisting of a metal, agarose, acrylic, and plastic. In some embodiments, the nanoparticle is magnetic. In some embodiments, the nanoparticle is conjugated to the peptide by a linker comprising a chemical linker, a peptide linker, or a combination thereof.

In some embodiments, the one or more capture molecules derived from the ACE2 receptor comprises an amino acid sequence that is at least 70%, 75%, 80%, 85%, 90%, 91%, 92%, 93%, 94%, 95%, 96%, 97%, 98%, 99%, 100% identical to SEQ ID NO: 1. In some embodiments, the peptide derived from a spike glycoprotein of a coronavirus comprises at least a portion of a spike protein derived from Severe acute respiratory syndrome-associated coronavirus (SARS-CoV). In some embodiments, the SARS-CoV is SARS-CoV-2. In some embodiments, an infection in a human subject by the SARS-CoV-2 causes coronavirus disease of 2019 (COVID-19) in the human subject. In some embodiments, the at least a portion of the spike protein comprises a subunit 1 of the spike protein. In some embodiments, the at least a portion of the spike protein comprises a receptor binding domain (RBD) of the subunit 1 of the spike protein. In some embodiments, the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 2. In some embodiments, the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 3. In some embodiments, the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 4. In some embodiments, the complex between the peptide-conjugate and the one or more capture molecules on the surface is visible on the surface using color, reflectance, fluorescence, bioluminescence, or chemiluminescence.

In some embodiments, systems further comprise a housing at least partially enclosing the surface. In some embodiments, systems further comprise a sample receptor configured to receive a biological sample from a subject. In some embodiments, the sample receptor is mechanically coupled to a housing at least partially enclosing the surface. In some embodiments, the biological sample comprises one or more antibodies specific to the peptide. In some embodiments, the biological sample does not consist of one or more antibodies specific to the peptide. In some embodiments, the one or more antibodies belong to one or more immunoglobulin classes comprising immunoglobulin M, immunoglobulin G, immunoglobulin A, immunoglobulin E, and immunoglobulin D. In some embodiments, the subject was, or is, exposed to the coronavirus. In some embodiments, exposure of the subject to the coronavirus is unknown. In some embodiments, the subject was administered a vaccine against the coronavirus. In some embodiments, the biological sample comprises blood, urine, saliva, or feces. In some embodiments, the blood is capillary blood. In some embodiments, systems further comprise a transdermal puncture device configured to obtain the capillary blood from the subject. In some embodiments, the sample receptor comprises a filter to separate serum from the blood.

In some embodiments, systems further comprise a data store for storing data from the image that is captured by the imaging device. In some embodiments, the data store is a cloud-based or a web-based data store, or a local data store. In some embodiments, the data comprises one or more of geolocation of the imaging device, a result from the image captured by the imaging device, and external data. In some embodiments, external data is data from an external device selected from a diagnostic device, a prognostic device, or a health or fitness tracking device. In some embodiments, external data comprises body temperature, heart rate variability, resting heart rate, sleep quality, and sleep quantity. In some embodiments, systems further comprise an external device selected from a diagnostic device, a prognostic device, or a health or fitness tracking device. In some embodiments, the imaging device is a personal electronic device. In some embodiments, the personal electronic device is a smart phone, tablet, body camera, web camera, or personal computer. In some embodiments, the personal electronic device comprises a web-based portal. In some embodiments, the web-based portal utilizes an application. In some embodiments, the application is configured to receive data comprising a result from the image captured by the personal electronic device, a geolocation of the personal electronic device, and external data from one or more external device. In some embodiments, the application comprises a data analytics module configured to analyze the result by: (a) determining whether the result is a positive result or a negative result, wherein a positive results indicates immunity and a negative results indicates a lack of immunity; (b) determining an absolute number of complexes between the peptide-conjugate and the ACE2 receptor on the surface; or (c) determining a level of binding between the peptide-conjugate and the ACE2, wherein a high level of binding indicates a low level of immunity, and a low level of binding indicates a high level of immunity. In some embodiments, the positive or the negative result is relative to a threshold number of complexes between the peptide-conjugate and the ACE2 receptor on the surface. In some embodiments, the threshold number is predetermined relative to an index a control. In some embodiments, the data analytics module is further configured to normalize the result by subtracting background noise. In some embodiments, the data analytics module is further configured to identify a geographical location comprised of subjects for which a positive result was determined to detect a presence of herd immunity to the coronavirus, or recommend further testing. In some embodiments, the data analytics module utilizes geofencing from coordinates of the personal electronic device to identify the geographical location. In some embodiments, the data analytics module utilizes a machine learning algorithm, artificial intelligence, or both.

Aspects disclosed herein comprise systems comprising: (a) one or more capture molecules derived from a spike glycoprotein of a coronavirus; (b) a peptide-conjugate comprising: (i) a peptide derived from angiotensin-converting enzyme 2 (ACE2) receptor; and (ii) a detectable moiety. In some embodiments, the system further comprises (a) a surface; and (b) an imaging device configured to capture an image of a complex between the peptide-conjugate and the one or more capture molecules on the surface when the complex is coupled to the surface. In some embodiments, the system further comprises an imaging device configured to capture an image of a complex between the peptide-conjugate and the one or more capture molecules. In some embodiments, the system further comprises a container comprising (a) and (b), wherein the container is portable.

In some embodiments, the system is a point of need system. In some embodiments, the point of need is a point of care system. In some embodiments, the surface comprises a material selected from the group consisting of a metal, a plastic, glass, and a nitrocellulose membrane. In some embodiments, the surface is a passivated surface. In some embodiments, the passivated surface comprises a polymer layer comprising a molecule selected from the group consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran. In some embodiments, the complex is coupled to the surface. In some embodiments, the complex is coupled to the surface by a covalent bond, a linker, or a combination thereof. In some embodiments, the linker is a chemical linker, a peptide linker, or a combination thereof. In some embodiments, the one or more capture molecules is coupled to the surface. In some embodiments, the one or more capture molecules is coupled to the surface by a covalent bond, a linker, or a combination thereof. In some embodiments, the linker is a chemical linker, a peptide linker, or a combination thereof. In some embodiments, the one or more capture molecules is a fusion polypeptide. In some embodiments, the fusion polypeptide comprises at least a portion of a fragment crystallizable region (Fc) region of a monoclonal antibody. In some embodiments, the one or more capture molecules is bound by an antibody that is coupled to the surface. In some embodiments, the peptide-conjugate comprises a nanoparticle, a fluorescent dye, an enzymatic label, or a colorimetric label, or a combination thereof. In some embodiments, the nanoparticle comprises a material selected from the group consisting of a metal, agarose, acrylic, and plastic. In some embodiments, the nanoparticle is magnetic. In some embodiments, the nanoparticle is conjugated to the peptide by a linker comprising a chemical linker, a peptide linker, or a combination thereof.

In some embodiments, the peptide derived from the ACE2 receptor comprises an amino acid sequence that is at least 70%, 75%, 80%, 85%, 90%, 91%, 92%, 93%, 94%, 95%, 96%, 97%, 98%, 99%, 100% identical to SEQ ID NO: 1. In some embodiments, the one or more capture molecules derived from a spike glycoprotein of a coronavirus comprises at least a portion of a spike protein derived from Severe acute respiratory syndrome-associated coronavirus (SARS-CoV). In some embodiments, the SARS-CoV is SARS-CoV-2. In some embodiments, an infection in a human subject by the SARS-CoV-2 causes coronavirus disease of 2019 (COVID-19) in the human subject. In some embodiments, the at least a portion of the spike protein comprises a subunit 1 of the spike protein. In some embodiments, the at least a portion of the spike protein comprises a receptor binding domain (RBD) of the subunit 1 of the spike protein.

In some embodiments, the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 2. In some embodiments, the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 3. In some embodiments, the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 4. In some embodiments, the complex between the peptide-conjugate and the one or more capture molecules on the surface is visible on the surface using color, reflectance, fluorescence, bioluminescence, or chemiluminescence.

In some embodiments, systems further comprise a housing at least partially enclosing the surface. In some embodiments, systems further comprise a sample receptor configured to receive a biological sample from a subject. In some embodiments, the sample receptor is mechanically coupled to a housing at least partially enclosing the surface. In some embodiments, the biological sample comprises one or more antibodies specific to the peptide. In some embodiments, the biological sample does not consist of one or more antibodies specific to the peptide. In some embodiments, the one or more antibodies belong to one or more immunoglobulin classes comprising immunoglobulin M, immunoglobulin G, immunoglobulin A, immunoglobulin E, and immunoglobulin D. In some embodiments, the subject was, or is, exposed to the coronavirus. In some embodiments, exposure of the subject to the coronavirus is unknown. In some embodiments, the subject was administered a vaccine against the coronavirus. In some embodiments, the biological sample comprises blood, urine, saliva, or feces. In some embodiments, the blood is capillary blood. In some embodiments, systems further comprise a transdermal puncture device configured to obtain the capillary blood from the subject. In some embodiments, the sample receptor comprises a filter to separate serum from the blood.

In some embodiments, systems further comprise a data store for storing data from the image that is captured by the imaging device. In some embodiments, the data store is a cloud-based or a web-based data store, or a local data store. In some embodiments, the data comprises one or more of geolocation of the imaging device, a result from the image captured by the imaging device, and external data. In some embodiments, external data is data from an external device selected from a diagnostic device, a prognostic device, or a health or fitness tracking device. In some embodiments, external data comprises body temperature, heart rate variability, resting heart rate, sleep quality, and sleep quantity. In some embodiments, systems further comprise an external device selected from a diagnostic device, a prognostic device, or a health or fitness tracking device. In some embodiments, the imaging device is a personal electronic device. In some embodiments, the personal electronic device is a smart phone, tablet, body camera, web camera, or personal computer. In some embodiments, the personal electronic device comprises a web-based portal. In some embodiments, the web-based portal utilizes an application. In some embodiments, the application is configured to receive data comprising a result from the image captured by the personal electronic device, a geolocation of the personal electronic device, and external data from one or more external device. In some embodiments, the application comprises a data analytics module configured to analyze the result by: (a) determining whether the result is a positive result or a negative result, wherein a positive results indicates immunity and a negative results indicates a lack of immunity; (b) determining an absolute number of complexes between the peptide-conjugate and the ACE2 receptor on the surface; or (c) determining a level of binding between the peptide-conjugate and the ACE2, wherein a high level of binding indicates a low level of immunity, and a low level of binding indicates a high level of immunity. In some embodiments, the positive or the negative result is relative to a threshold number of complexes between the peptide-conjugate and the ACE2 receptor on the surface. In some embodiments, the threshold number is predetermined relative to an index a control. In some embodiments, the data analytics module is further configured to normalize the result by subtracting background noise. In some embodiments, the data analytics module is further configured to identify a geographical location comprised of subjects for which a positive result was determined to detect a presence of herd immunity to the coronavirus, or recommend further testing. In some embodiments, the data analytics module utilizes geofencing from coordinates of the personal electronic device to identify the geographical location. In some embodiments, the data analytics module utilizes a machine learning algorithm, artificial intelligence, or both.

Aspects disclosed herein provide methods of identifying adaptive immunity to a coronavirus in a subject, the method comprising: (a) obtaining a biological sample from the subject; (b) producing a mixture by introducing the biological sample with a detectable peptide derived from a spike glycoprotein of a coronavirus; (c) bringing the mixture into contact with one or more capture molecules derived from an angiotensin-converting enzyme 2 (ACE2) receptor; (d) detecting a number of binding complexes between the detectable peptide and the one or more capture molecules; (e) if the number of the binding complexes is low relative to an index or a control, then identifying the subject as being immune to an infection by the coronavirus; and (f) if the number of the binding complexes is high relative to an index or a control, then identifying the subject as not being immune to an infection by the coronavirus.

In some embodiments, steps (a)-(f) are performed at the point of need. In some embodiments, steps (a)-(f) are performed at the point of care. In some embodiments, the surface comprises a material selected from the group consisting of a metal, a plastic, glass, and a nitrocellulose membrane. In some embodiments, the surface is a passivated surface. In some embodiments, the passivated surface comprises a polymer layer comprising a molecule selected from the group consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran. In some embodiments, the one or more capture molecules is coupled to the surface by a covalent bond, a linker, or a combination thereof. In some embodiments, the linker is a chemical linker, a peptide linker, or a combination thereof. In some embodiments, the one or more capture molecules is a fusion polypeptide. In some embodiments, the fusion polypeptide comprises at least a portion of a fragment crystallizable region (Fc) region of a monoclonal antibody. In some embodiments, the one or more capture molecules is bound by an antibody that is coupled to the surface. In some embodiments, the detectable peptide comprises a nanoparticle, a fluorescent dye, an enzymatic label, or a colorimetric label, or a combination thereof. In some embodiments, the nanoparticle comprises a material selected from the group consisting of a metal, agarose, acrylic, and plastic. In some embodiments, the nanoparticle is magnetic. In some embodiments, the nanoparticle is conjugated to the peptide by a linker comprising a chemical linker, a peptide linker, or a combination thereof.

In some embodiments, the detectable peptide derived from the ACE2 receptor comprises an amino acid sequence that is at least 70%, 75%, 80%, 85%, 90%, 91%, 92%, 93%, 94%, 95%, 96%, 97%, 98%, 99%, 100% identical to SEQ ID NO: 1. In some embodiments, the one or more capture molecules derived from the spike glycoprotein of a coronavirus comprises at least a portion of a spike protein derived from Severe acute respiratory syndrome-associated coronavirus (SARS-CoV). In some embodiments, the SARS-CoV is SARS-CoV-2. In some embodiments, an infection in a human subject by the SARS-CoV-2 causes coronavirus disease of 2019 (COVID-19) in the human subject. In some embodiments, the at least a portion of the spike protein comprises a subunit 1 of the spike protein. In some embodiments, the at least a portion of the spike protein comprises a receptor binding domain (RBD) of the subunit 1 of the spike protein. In some embodiments, the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 2. In some embodiments, the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 3. In some embodiments, the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 4. In some embodiments, the complex between the peptide-conjugate and the one or more capture molecules is visible on the surface using color, reflectance, fluorescence, bioluminescence, or chemiluminescence.

In some embodiments, the one or more capture molecules derived from the ACE2 receptor comprises an amino acid sequence that is at least 70%, 75%, 80%, 85%, 90%, 91%, 92%, 93%, 94%, 95%, 96%, 97%, 98%, 99%, 100% identical to SEQ ID NO: 1. In some embodiments, the detectable peptide derived from the spike glycoprotein of a coronavirus comprises at least a portion of a spike protein derived from Severe acute respiratory syndrome-associated coronavirus (SARS-CoV). In some embodiments, the SARS-CoV is SARS-CoV-2. In some embodiments, an infection in a human subject by the SARS-CoV-2 causes coronavirus disease of 2019 (COVID-19) in the human subject. In some embodiments, the at least a portion of the spike protein comprises a subunit 1 of the spike protein. In some embodiments, the at least a portion of the spike protein comprises a receptor binding domain (RBD) of the subunit 1 of the spike protein. In some embodiments, the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 2. In some embodiments, the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 3. In some embodiments, the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 4. In some embodiments, the complex between the peptide-conjugate and the one or more capture molecules is visible on the surface using color, reflectance, fluorescence, bioluminescence, or chemiluminescence.

In some embodiments, methods further comprise providing a web-based portal on the personal electronic device. In some embodiments, methods further comprise providing an application on the web-based portal. In some embodiments, methods further comprise receiving data, by the application, the data comprising a result from the image captured by the personal electronic device, a geolocation of the personal electronic device, or external data from one or more external devices. In some embodiments, methods further comprise providing a data analytics module at the application. In some embodiments, methods further comprise analyzing the result, by the data analytics module, to determining whether the result is a positive result or a negative result, wherein a positive results indicates immunity and a negative results indicates a lack of immunity. In some embodiments, the positive or the negative result is relative to a threshold number of complexes between the detectable peptide and the one or more capture molecules on the surface. In some embodiments, the threshold number is predetermined relative to an index a control. In some embodiments, methods further comprise analyzing the result, by the data analytics module, to determining an absolute number of complexes between the detectable peptide and the one or more capture molecules on the surface. In some embodiments, methods further comprise analyzing the result, by the data analytics module, to determine a level of binding between the peptide-conjugate and the ACE2, wherein a high level of binding indicates a low level of immunity, and a low level of binding indicates a high level of immunity. In some embodiments, methods further comprise normalizing, by the data analytics module, the result by subtracting background noise. In some embodiments, methods further comprise identifying, by the data analytics module, a geographical location comprised of subjects for which a positive result was determined to detect a presence of herd immunity to the coronavirus, or recommend further testing. In some embodiments, identifying the geographical location comprises utilizing geofencing from coordinates of the personal electronic device. In some embodiments, the data analytics module utilizes a machine learning algorithm, artificial intelligence, or both. In some embodiments, methods further comprise providing a data store that is a cloud-based data store or a web-based data store, or a local data store. In some embodiments, the data comprises one or more of geolocation of the imaging device, a result from the image captured by the imaging device, and external data. In some embodiments, external data is data from an external device selected from a diagnostic device, a prognostic device, or a health or fitness tracking device. In some embodiments, external data comprises body temperature, heart rate variability, resting heart rate, sleep quality, and sleep quantity. In some embodiments, comprising receiving, from an external device, the external data, wherein the external device is selected from a diagnostic device, a prognostic device, or a health or fitness tracking device.

Aspects disclosed herein provide methods of identifying adaptive immunity to a coronavirus in a subject, the method comprising: (a) obtaining a biological sample from the subject; (b) producing a mixture by introducing the biological sample with a detectable peptide derived from an angiotensin-converting enzyme 2 (ACE2) receptor; (c) bringing the mixture into contact with one or more capture molecules derived from a spike glycoprotein of a coronavirus; (d) detecting a number of binding complexes between the detectable peptide and the one or more capture molecules; (e) if the number of the binding complexes is low relative to an index or a control, then identifying the subject as being immune to an infection by the coronavirus; and (f) if the number of the binding complexes is high relative to an index or a control, then identifying the subject as not being immune to an infection by the coronavirus.

In some embodiments, steps (a)-(f) are performed at the point of need. In some embodiments, steps (a)-(f) are performed at the point of care. In some embodiments, the surface comprises a material selected from the group consisting of a metal, a plastic, glass, and a nitrocellulose membrane. In some embodiments, the surface is a passivated surface. In some embodiments, the passivated surface comprises a polymer layer comprising a molecule selected from the group consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran. In some embodiments, the one or more capture molecules is coupled to the surface by a covalent bond, a linker, or a combination thereof. In some embodiments, the linker is a chemical linker, a peptide linker, or a combination thereof. In some embodiments, the one or more capture molecules is a fusion polypeptide. In some embodiments, the fusion polypeptide comprises at least a portion of a fragment crystallizable region (Fc) region of a monoclonal antibody. In some embodiments, the one or more capture molecules is bound by an antibody that is coupled to the surface. In some embodiments, the detectable peptide comprises a nanoparticle, a fluorescent dye, an enzymatic label, or a colorimetric label, or a combination thereof. In some embodiments, the nanoparticle comprises a material selected from the group consisting of a metal, agarose, acrylic, and plastic. In some embodiments, the nanoparticle is magnetic. In some embodiments, the nanoparticle is conjugated to the peptide by a linker comprising a chemical linker, a peptide linker, or a combination thereof.

In some embodiments, the detectable peptide derived from the ACE2 receptor comprises an amino acid sequence that is at least 70%, 75%, 80%, 85%, 90%, 91%, 92%, 93%, 94%, 95%, 96%, 97%, 98%, 99%, 100% identical to SEQ ID NO: 1. In some embodiments, the one or more capture molecules derived from the spike glycoprotein of a coronavirus comprises at least a portion of a spike protein derived from Severe acute respiratory syndrome-associated coronavirus (SARS-CoV). In some embodiments, the SARS-CoV is SARS-CoV-2. In some embodiments, an infection in a human subject by the SARS-CoV-2 causes coronavirus disease of 2019 (COVID-19) in the human subject. In some embodiments, the at least a portion of the spike protein comprises a subunit 1 of the spike protein. In some embodiments, the at least a portion of the spike protein comprises a receptor binding domain (RBD) of the subunit 1 of the spike protein. In some embodiments, the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 2. In some embodiments, the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 3. In some embodiments, the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 4. In some embodiments, the complex between the peptide-conjugate and the one or more capture molecules is visible on the surface using color, reflectance, fluorescence, bioluminescence, or chemiluminescence.

In some embodiments, the biological sample comprises one or more antibodies specific to the peptide. In some embodiments, the biological sample does not consist of one or more antibodies specific to the peptide. In some embodiments, the one or more antibodies belong to one or more immunoglobulin classes comprising immunoglobulin M, immunoglobulin G, immunoglobulin A, immunoglobulin E, and immunoglobulin D. In some embodiments, the subject was, or is, exposed to the coronavirus. In some embodiments, exposure of the subject to the coronavirus is unknown. In some embodiments, the subject is a plurality of subjects. In some embodiments, methods further comprise identifying adaptive immunity of the plurality of subjects to the coronavirus. In some embodiments, methods further comprise monitoring a spread of infection of the plurality of subjects by the coronavirus. In some embodiments, the subject was administered a vaccine against the coronavirus. In some embodiments, methods further comprise determining that the vaccine is effective to substantially immunize the subject against the coronavirus, provided the number of the binding complexes is low relative to an index or a control. In some embodiments, the biological sample comprises blood, urine, saliva, or feces. In some embodiments, the blood is capillary blood. In some embodiments, the capillary blood is obtained from the subject by a prick of the subject's finger. In some embodiments, methods further comprise separating serum from the blood in the biological sample. In some embodiments, detecting in (d) comprises capturing an image of the surface with an imaging device to detect a number of binding complexes between the detectable peptide and the one or more capture molecules. In some embodiments, the imaging device is a personal electronic device. In some embodiments, the personal electronic device is a smart phone, tablet, body camera, web camera, or personal computer.

In some embodiments, methods further comprise providing a web-based portal on the personal electronic device. In some embodiments, methods further comprise providing an application on the web-based portal. In some embodiments, methods further comprise receiving data, by the application, the data comprising a result from the image captured by the personal electronic device, a geolocation of the personal electronic device, or external data from one or more external devices. In some embodiments, methods further comprise providing a data analytics module at the application. In some embodiments, methods further comprise analyzing the result, by the data analytics module, to determining whether the result is a positive result or a negative result, wherein a positive results indicates immunity and a negative results indicates a lack of immunity. In some embodiments, the positive or the negative result is relative to a threshold number of complexes between the detectable peptide and the one or more capture molecules on the surface. In some embodiments, the threshold number is predetermined relative to an index a control. In some embodiments, methods further comprise analyzing the result, by the data analytics module, to determining an absolute number of complexes between the detectable peptide and the one or more capture molecules on the surface. In some embodiments, methods further comprise analyzing the result, by the data analytics module, to determine a level of binding between the peptide-conjugate and the ACE2, wherein a high level of binding indicates a low level of immunity, and a low level of binding indicates a high level of immunity. In some embodiments, methods further comprise normalizing, by the data analytics module, the result by subtracting background noise. In some embodiments, methods further comprise identifying, by the data analytics module, a geographical location comprised of subjects for which a positive result was determined to detect a presence of herd immunity to the coronavirus, or recommend further testing. In some embodiments, identifying the geographical location comprises utilizing geofencing from coordinates of the personal electronic device. In some embodiments, the data analytics module utilizes a machine learning algorithm, artificial intelligence, or both. In some embodiments, methods further comprise providing a data store that is a cloud-based data store or a web-based data store, or a local data store. In some embodiments, the data comprises one or more of geolocation of the imaging device, a result from the image captured by the imaging device, and external data. In some embodiments, external data is data from an external device selected from a diagnostic device, a prognostic device, or a health or fitness tracking device. In some embodiments, external data comprises body temperature, heart rate variability, resting heart rate, sleep quality, and sleep quantity. In some embodiments, comprising receiving, from an external device, the external data, wherein the external device is selected from a diagnostic device, a prognostic device, or a health or fitness tracking device.

Aspects disclosed herein provide devices comprising: (a) a liquid composition comprising a peptide-conjugate comprising: (i) a peptide derived from a spike glycoprotein of a coronavirus; and (ii) a detectable moiety; and (b) a surface submerged in the liquid composition, the surface comprising one or more capture molecules coupled to the surface, the one or more capture molecules derived from an angiotensin-converting enzyme 2 (ACE2) receptor.

In some embodiments, the surface is a surface of a container, wherein the container contains (a) and (b). In some embodiments, the device is portable. In some embodiments, the device is a point of need device. In some embodiments, the point of need is a point of care. In some embodiments, the surface comprises a material selected from the group consisting of a metal, a plastic, glass, and a nitrocellulose membrane. In some embodiments, the surface is a passivated surface. In some embodiments, the passivated surface comprises a polymer layer comprising a molecule selected from the group consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran. In some embodiments, the one or more capture molecules is coupled to the surface by a covalent bond, a linker, or a combination thereof. In some embodiments, the linker is a chemical linker, a peptide linker, or a combination thereof. In some embodiments, the one or more capture molecules is a fusion polypeptide. In some embodiments, the fusion polypeptide comprises at least a portion of a fragment crystallizable region (Fc) region of a monoclonal antibody. In some embodiments, the one or more capture molecules is bound by an antibody that is coupled to the surface. In some embodiments, the peptide-conjugate comprises a nanoparticle, a fluorescent dye, an enzymatic label, or a colorimetric label, or a combination thereof. In some embodiments, the nanoparticle comprises a material selected from the group consisting of a metal, agarose, acrylic, and plastic. In some embodiments, the nanoparticle is magnetic. In some embodiments, the nanoparticle is conjugated to the peptide by a linker comprising a chemical linker, a peptide linker, or a combination thereof.

In some embodiments, the one or more capture molecules derived from the ACE2 receptor comprises an amino acid sequence that is at least 70%, 75%, 80%, 85%, 90%, 91%, 92%, 93%, 94%, 95%, 96%, 97%, 98%, 99%, 100% identical to SEQ ID NO: 1. In some embodiments, the peptide derived from the spike glycoprotein of a coronavirus comprises at least a portion of a spike protein derived from Severe acute respiratory syndrome-associated coronavirus (SARS-CoV). In some embodiments, the SARS-CoV is SARS-CoV-2. In some embodiments, an infection in a human subject by the SARS-CoV-2 causes coronavirus disease of 2019 (COVID-19) in the human subject. In some embodiments, the at least a portion of the spike protein comprises a subunit 1 of the spike protein. In some embodiments, the at least a portion of the spike protein comprises a receptor binding domain (RBD) of the subunit 1 of the spike protein. In some embodiments, the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 2. In some embodiments, the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 3. In some embodiments, the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 4. In some embodiments, the complex between the peptide-conjugate and the one or more capture molecules receptor on the surface is visible on the surface using color, reflectance, fluorescence, bioluminescence, or chemiluminescence.

In some embodiments, devices further comprise a housing at least partially enclosing the surface. In some embodiments, devices further comprise a sample receptor configured to receive a biological sample from a subject. In some embodiments, the sample receptor is mechanically coupled to a housing at least partially enclosing the surface. In some embodiments, the biological sample comprises one or more antibodies specific to the peptide. In some embodiments, the biological sample does not consist of one or more antibodies specific to the peptide. In some embodiments, the one or more antibodies belong to one or more immunoglobulin classes comprising immunoglobulin M, immunoglobulin G, immunoglobulin A, immunoglobulin E, and immunoglobulin D. In some embodiments, the subject was, or is, exposed to the coronavirus. In some embodiments, exposure of the subject to the coronavirus is unknown. In some embodiments, the subject was administered a vaccine against the coronavirus. In some embodiments, the biological sample comprises blood, urine, saliva, or feces. In some embodiments, the blood is capillary blood. In some embodiments, devices further comprising a transdermal puncture device configured to obtain the capillary blood from the subject. In some embodiments, the sample receptor comprises a filter to separate serum from the blood. In some embodiments, the device is a single integrated device.

Aspects disclosed herein provide devices comprising: (a) a liquid composition comprising a peptide-conjugate comprising: (i) a peptide derived from an angiotensin-converting enzyme 2 (ACE2) receptor; and (ii) a detectable moiety; and (b) a surface submerged in the liquid composition, the surface comprising one or more capture molecules coupled to the surface, the one or more capture molecules derived from a spike glycoprotein of a coronavirus.

In some embodiments, the surface is a surface of a container, wherein the container contains (a) and (b). In some embodiments, the device is portable. In some embodiments, the device is a point of need device. In some embodiments, the point of need is a point of care. In some embodiments, the surface comprises a material selected from the group consisting of a metal, a plastic, glass, and a nitrocellulose membrane. In some embodiments, the surface is a passivated surface. In some embodiments, the passivated surface comprises a polymer layer comprising a molecule selected from the group consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran. In some embodiments, the one or more capture molecules is coupled to the surface by a covalent bond, a linker, or a combination thereof. In some embodiments, the linker is a chemical linker, a peptide linker, or a combination thereof. In some embodiments, the one or more capture molecules is a fusion polypeptide. In some embodiments, the fusion polypeptide comprises at least a portion of a fragment crystallizable region (Fc) region of a monoclonal antibody. In some embodiments, the one or more capture molecules is bound by an antibody that is coupled to the surface. In some embodiments, the peptide-conjugate comprises a nanoparticle, a fluorescent dye, an enzymatic label, or a colorimetric label, or a combination thereof. In some embodiments, the nanoparticle comprises a material selected from the group consisting of a metal, agarose, acrylic, and plastic. In some embodiments, the nanoparticle is magnetic. In some embodiments, the nanoparticle is conjugated to the peptide by a linker comprising a chemical linker, a peptide linker, or a combination thereof.

In some embodiments, the peptide derived from the ACE2 receptor comprises an amino acid sequence that is at least 70%, 75%, 80%, 85%, 90%, 91%, 92%, 93%, 94%, 95%, 96%, 97%, 98%, 99%, 100% identical to SEQ ID NO: 1. In some embodiments, the one or more capture molecules derived from the spike glycoprotein of a coronavirus comprises at least a portion of a spike protein derived from Severe acute respiratory syndrome-associated coronavirus (SARS-CoV). In some embodiments, the SARS-CoV is SARS-CoV-2. In some embodiments, an infection in a human subject by the SARS-CoV-2 causes coronavirus disease of 2019 (COVID-19) in the human subject. In some embodiments, the at least a portion of the spike protein comprises a subunit 1 of the spike protein. In some embodiments, the at least a portion of the spike protein comprises a receptor binding domain (RBD) of the subunit 1 of the spike protein. In some embodiments, the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 2. In some embodiments, the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 3. In some embodiments, the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 4. In some embodiments, the complex between the peptide-conjugate and the one or more capture molecules receptor on the surface is visible on the surface using color, reflectance, fluorescence, bioluminescence, or chemiluminescence.

In some embodiments, devices further comprise a housing at least partially enclosing the surface. In some embodiments, devices further comprise a sample receptor configured to receive a biological sample from a subject. In some embodiments, the sample receptor is mechanically coupled to a housing at least partially enclosing the surface. In some embodiments, the biological sample comprises one or more antibodies specific to the peptide. In some embodiments, the biological sample does not consist of one or more antibodies specific to the peptide. In some embodiments, the one or more antibodies belong to one or more immunoglobulin classes comprising immunoglobulin M, immunoglobulin G, immunoglobulin A, immunoglobulin E, and immunoglobulin D. In some embodiments, the subject was, or is, exposed to the coronavirus. In some embodiments, exposure of the subject to the coronavirus is unknown. In some embodiments, the subject was administered a vaccine against the coronavirus. In some embodiments, the biological sample comprises blood, urine, saliva, or feces. In some embodiments, the blood is capillary blood. In some embodiments, devices further comprising a transdermal puncture device configured to obtain the capillary blood from the subject. In some embodiments, the sample receptor comprises a filter to separate serum from the blood. In some embodiments, the device is a single integrated device.

Aspects disclosed herein provide methods of using the device of the present disclosure, the method comprising (a) receiving, by the sample receptor, a biological sampling from a subject; (b) adding the biological sample to the liquid composition comprising the peptide-conjugate; (c) applying the liquid composition to the surface, thereby submerging the surface with the liquid composition; and (d) detecting a presence of binding between the one or more capture molecules and the peptide-conjugate, or (e) detecting an absence of binding between the one or more capture molecules and the peptide-conjugate. In some embodiments, methods comprise detecting a presence of binding between the one or more capture molecules and the peptide-conjugate, indicating that the subject is not immune to an infection by a pathogen mediated by an interaction between the peptide of the peptide conjugate and the one or more capture molecules in vivo. In some embodiments, methods comprise detecting an absence of binding between the one or more capture molecules and the peptide-conjugate, indicating that the subject is immune to an infection by a pathogen mediated by an interaction between the peptide of the peptide conjugate and the one or more capture molecules in vivo. In some embodiments, steps (a)-(w) are performed at the point of need. In some embodiments, steps (a)-(e) are performed at the point of care.

In some embodiments, the subject was, or is, exposed to the coronavirus. In some embodiments, exposure of the subject to the coronavirus is unknown. In some embodiments, the subject is a plurality of subjects. In some embodiments, methods further comprise identifying adaptive immunity of the plurality of subjects to the coronavirus. In some embodiments, methods further comprise monitoring a spread of infection of the plurality of subjects by the coronavirus. In some embodiments, the subject was administered a vaccine against the coronavirus. In some embodiments, methods further comprise determining that the vaccine is effective to substantially immunize the subject against the coronavirus, provided the number of the binding complexes is low relative to an index or a control. In some embodiments, the biological sample comprises blood, urine, saliva, or feces. In some embodiments, the blood is capillary blood. In some embodiments, the capillary blood is obtained from the subject by a prick of the subject's finger. In some embodiments, methods further comprise separating serum from the blood in the biological sample.

In some embodiments, methods further comprise providing a web-based portal on the personal electronic device. In some embodiments, methods further comprise providing an application on the web-based portal. In some embodiments, methods further comprise receiving data, by the application, the data comprising a result from the image captured by the personal electronic device, a geolocation of the personal electronic device, or external data from one or more external devices. In some embodiments, methods further comprise providing a data analytics module at the application. In some embodiments, methods further comprise analyzing the result, by the data analytics module, to determining whether the result is a positive result or a negative result, wherein a positive results indicates immunity and a negative results indicates a lack of immunity. In some embodiments, the positive or the negative result is relative to a threshold number of complexes between the detectable peptide and the one or more capture molecules on the surface. In some embodiments, the threshold number is predetermined relative to an index a control. In some embodiments, methods further comprise analyzing the result, by the data analytics module, to determining an absolute number of complexes between the detectable peptide and the one or more capture molecules on the surface. In some embodiments, methods further comprise analyzing the result, by the data analytics module, to determine a level of binding between the peptide-conjugate and the ACE2, wherein a high level of binding indicates a low level of immunity, and a low level of binding indicates a high level of immunity. In some embodiments, methods further comprise normalizing, by the data analytics module, the result by subtracting background noise. In some embodiments, methods further comprise identifying, by the data analytics module, a geographical location comprised of subjects for which a positive result was determined to detect a presence of herd immunity to the coronavirus, or recommend further testing. In some embodiments, identifying the geographical location comprises utilizing geofencing from coordinates of the personal electronic device. In some embodiments, the data analytics module utilizes a machine learning algorithm, artificial intelligence, or both. In some embodiments, methods further comprise providing a data store that is a cloud-based data store or a web-based data store, or a local data store. In some embodiments, the data comprises one or more of geolocation of the imaging device, a result from the image captured by the imaging device, and external data. In some embodiments, external data is data from an external device selected from a diagnostic device, a prognostic device, or a health or fitness tracking device. In some embodiments, external data comprises body temperature, heart rate variability, resting heart rate, sleep quality, and sleep quantity. In some embodiments, comprising receiving, from an external device, the external data, wherein the external device is selected from a diagnostic device, a prognostic device, or a health or fitness tracking device.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an exemplary assay measuring binding between an immobilized human angiotensin-converting enzyme 2 (ACE2) receptor and a peptide-conjugate derived from a spike glycoprotein of a SARS-CoV-2 in a biological sample obtained from a patient that has not been exposed to SARS-CoV-2.

FIG. 2 shows an exemplary assay measuring binding between an immobilized human ACE2 receptor and a peptide-conjugate derived from the spike glycoprotein of SARS-CoV-2 in a biological sample obtained from a patient exposed to SARS-CoV-2.

FIG. 3 shows an exemplary lateral flow assay to measure binding between human ACE2 receptor and a peptide-conjugate derived from the spike glycoprotein of SARS-CoV-2 in a biological sample obtained from a patient that has not been exposed to SARS-CoV-2.

FIG. 4 shows an exemplary lateral flow assay to measure binding between human ACE2 receptor and a peptide-conjugate derived from the spike glycoprotein of SARS-CoV-2 in a biological sample obtained from a patient that was exposed to SARS-CoV-2.

FIG. 5 shows an exemplary system according to some embodiments.

FIG. 6 shows a computing device; in this case, a device with one or more processors, memory, storage, and a network interface, in accordance with some embodiments.

FIG. 7 shows an exemplary assay in FIG. 2 in an image-free system; results from the exemplary assay is visible and can be interpreted by the naked eye.

### DETAILED DESCRIPTION

Provided herein are testing devices and systems for measuring adaptive immunity to a pathogen in a subject. In some embodiments, the testing devices are point of need or point of care devices. In some embodiments, the testing devices are configured to perform an assay to detect antibodies against a pathogenic antigen in a biological sample of the subject. In some embodiments, the assay is a competition assay comprising one or more capture molecules coupled to a surface and a detectable peptide-conjugate. In some embodiments, a signal from the detectable peptide-conjugate is detected by capturing an image of a detection zone of the device by an imaging device. In some embodiments, the imaging device is a smartphone. In some embodiments, the assay is a lateral flow assay (LFA).

In some embodiments, the detectable peptide-conjugate comprises a peptide derived from a spike glycoprotein of a coronavirus, and the one or more capture molecules is derived from an angiotensin-converting enzyme 2 (ACE2) receptor. In some cases, the detectable peptide-conjugate comprises a peptide derived from the ACE2 receptor, and the one or more capture molecules is derived from the spike glycoprotein of a coronavirus. In some embodiments, the coronavirus is a severe acute respiratory syndrome coronavirus (SARS-CoV). In some embodiments, the SARS-CoV is SARS-CoV-2. In some embodiments, the ACE2 receptor is derived from a human ACE2 receptor.

Systems described herein comprise the testing device and the imaging device. In some embodiments, systems further comprise a computing device with an application (e.g., web or mobile) comprising a data analytics module for receiving an analyzing data from the imaging device to provide a result. In some embodiments, the imaging device is the computing device (e.g., smartphone). In some embodiments, the imaging device is not the computing device. In some embodiments, the result is a positive result indicating that a subject is immune to an infection by the pathogen. In some embodiments, the result is a negative result indicating that the subject is not immune to an infection by the pathogen. In some embodiments, the application further comprises a communication module configured to display via a graphical user interface (GUI) one or more results to a user. In some embodiments, systems comprise a data store configured to store and retrieve data from the imaging device, the external device, or both.

In some embodiments, systems comprise an external device, such as a wearable tracking device (e.g., Aurora®, Fitbit®, Apple Watch). In some embodiments, the data analytics module receives and analyzes external data from the external device. In some embodiments, external data comprise body temperature, heart rate, heart rate variability, sleep quality, or sleep quantify of the subject. In some embodiments, data analytics module is configured to analyze the external data in combination with the data received from the imaging device to identify the subject as being infected with a specific pathogen.

In some embodiments, systems described herein comprise multiple users with multiple computing devices. Within a geographical location of interest, systems described herein are designed to determine whether a population of users has become immune to an infection by the pathogen (e.g., SARS-CoV-2). In some instances, global position system (GPS) tracking of the personal computing device, the imaging devices, or both, can be used to produce a geofence surrounding the geographical location of interest.

### I. Testing Devices

Disclosed herein, in some embodiments, are testing devices that can be deployed at the point of need to determine whether a patient is immune to an infection by the pathogen. In some embodiments, devices comprise an assay assembly capable of assaying a biological sample obtained from the patient. In some embodiments, the testing devices comprise one or more components, such as a housing, a sample receiver, a sample processor, a sample purifier, or a detection zone.

### Assay Assembly

Disclosed herein, in some embodiments are devices comprising an assay assembly that is capable of detecting a target analyte. In some embodiments, the target analyte is an antibody specific to a pathogen of interest. In some embodiments, the antibody specific to the pathogen of interest functionally blocks binding between the pathogen to its cognate receptor. In some embodiments, the pathogen comprises a virus, a bacteria, a parasite, a fugus, or a combination thereof. In some embodiments, the virus is a coronavirus. In some embodiments, the coronavirus is a severe acute respiratory syndrome coronavirus (SARS-CoV). In some embodiments, the SARS-CoV is SARS-CoV-2. In some embodiments, the coronavirus is Middle East Respiratory Syndrome coronavirus (MERS-CoV). In some embodiments, the coronavirus is an alpha coronavirus (e.g., 229E, NL63). In some embodiments, the coronavirus is a beta coronavirus (e.g., OC43, HKU1). In some embodiments, the antibody specific to coronavirus is specific to the receptor binding domain of the spike protein of the coronavirus, such that the antibody blocks binding of the spike protein to its cognate receptor (e.g., ACE2). In some embodiments, the analyte is a complex comprising the spike protein bound to the antibody at the receptor binding region of the spike protein. In some embodiments, the antibody specific to the pathogen of interest belongs to an immunoglobulin class comprising immunoglobulin M, immunoglobulin G, immunoglobulin A, immunoglobulin E, or immunoglobulin D.

In some embodiments, the assay assembly comprises one or more capture molecules coupled to a solid surface. In some embodiments, the one or more capture molecules is derived from an angiotensin-converting enzyme 2 (ACE2) receptor. In some embodiments, ACE2 is human ACE2 (Entrez ID 59272). In some embodiments, the one or more capture molecules comprises a portion of the ACE2 polypeptide. In some embodiments, the one or more capture molecules comprises an amino acid sequence least 70%, 75%, 80%, 85%, 86%, 87%, 88%, 89%, 90%, 91%, 92%, 93%, 94%, 95%, 96%, 97%, 98%, 99%, or 100% identical to the human ACE2 polypeptide. In some embodiments, the amino acid sequence encoding the human ACE2 polypeptide is provided in SEQ ID NO: 1.

In some embodiments, the one or more capture molecules is derived from a spike glycoprotein of a coronavirus. In some embodiments, the coronavirus is a severe acute respiratory syndrome coronavirus (SARS-CoV). In some embodiments, the SARS-CoV is SARS-CoV-2. In some embodiments, the coronavirus is Middle East Respiratory Syndrome coronavirus (MERS-CoV). In some embodiments, the coronavirus is an alpha coronavirus (e.g., 229E, NL63). In some embodiments, the coronavirus is a beta coronavirus (e.g., OC43, HKU1). In some embodiments, the one or more capture molecules comprises a portion of the spike protein. In some embodiments, the portion comprises subunit 1 of the spike protein. In some embodiments, the portion comprises the receptor binding domain (RBD) of subunit 1 of the spike protein. In some embodiments, the one or more capture molecules comprises an amino acid sequence least 70%, 75%, 80%, 85%, 86%, 87%, 88%, 89%, 90%, 91%, 92%, 93%, 94%, 95%, 96%, 97%, 98%, 99%, or 100% identical to the spike protein, the subunit 1 of the spike protein, or the RBD of the subunit 1 of the spike protein, or a combination thereof. In some embodiments, the amino acid sequence encoding the spike protein is provided in SEQ ID NO: 2. In some embodiments, the amino acid sequence encoding the subunit 1 of the spike protein is provided in SEQ ID NO: 3. In some embodiments, the amino acid sequence encoding the RBD of the subunit 1 of the spike protein is provided in SEQ ID NO: 4.

In some embodiments, the assay assembly comprises a solid surface. In some embodiments, the solid surface is made of a metal, a plastic, glass, or a membrane. In some embodiments, the surface is passivated. In some embodiments, the surface comprises a polymer coating comprising a polymer selected from polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran.

In some embodiments, the one or more capture molecules is coupled to the surface directly or indirectly. Capture molecules coupled indirectly to the surface may, for example, be coupled to the surface by a linker. In some embodiments, the linker is a chemical linker, a peptide link, a polymer linker, or a combination thereof. In some embodiments, the one or more capture molecules is bound by a primary capture antibody that is bound to the surface covalently or non-covalently. Capture molecules coupled directly to the surface may, for example, be covalently or non-covalently bound to the surface.

In some embodiments, the one or more capture molecules is a fusion protein comprising a peptide directly or indirectly bound to the surface. In some embodiments, the peptide comprises a fragment crystallizable (Fc) region of a monoclonal antibody. In some embodiments, the Fc region is derived from an antibody belonging to an immunoglobulin class comprising immunoglobulin M, immunoglobulin G, immunoglobulin A, immunoglobulin E, or immunoglobulin D.

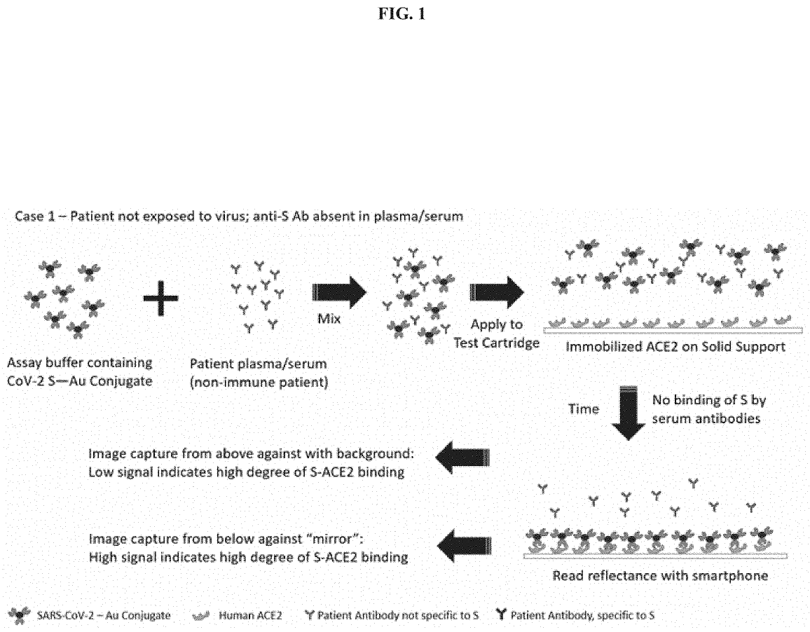

Referring to FIG. 1, a biological sample obtained from a subject that has not been exposed to a coronavirus is assayed using the assay assembly described herein. In some embodiments, a labeled capture molecule (“CoV-2 S—Au”) comprises a peptide-conjugate comprising a detection agent (e.g., gold nanoparticle) and a peptide derived from the spike protein of SARS-CoV-2. A fluid formulation comprising the peptide-conjugate is contacted with a biological sample from the subject. In this example, the target analyte is an activity of one or more antibodies comprising blocking the binding between the spike protein of SARS-CoV-2 and ACE2. In this example the patient has not been exposed to the coronavirus, so a presence of the analyte is not expected to be detected.

In some embodiments, an immobilized capture molecule is coupled to a solid surface. In some embodiments, the immobilized capture molecule is human ACE2 receptor. The liquid formulation is applied to the solid surface. A high number of complexes between the immobilized human ACE2 and the detectable peptide-conjugate is detected, correlating with an absence or a low amount of analyte in the sample. In some embodiments, an image of the surface is captured with an imaging device. In some embodiments, the image is a video or still image. In some embodiments, the imaging device comprises a reflectance reader. In some embodiments, the imaging device is a personal electronic device, such as a smartphone. When imaged from above the surface, a low signal indicates a high degree of binding between human ACE2 and the peptide-conjugate (therefore, a low amount of the analyte). When imaged from below the surface, a high signal indicates a high degree of binding between the human ACE2 and the peptide-conjugate.

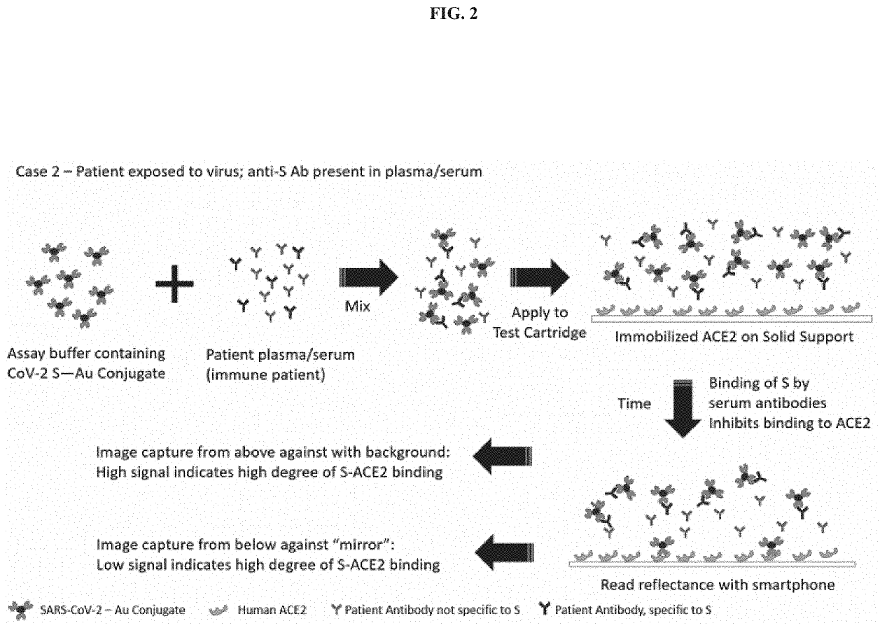

In contrast, a biological sample obtained from a subject that was exposed to a coronavirus assayed using the assay assembly described herein, is provided in FIG. 2. A low number of complexes between the immobilized human ACE2 and the detectable peptide-conjugate is detected, which correlates with a presence, absence, or a high amount of analyte in the sample.

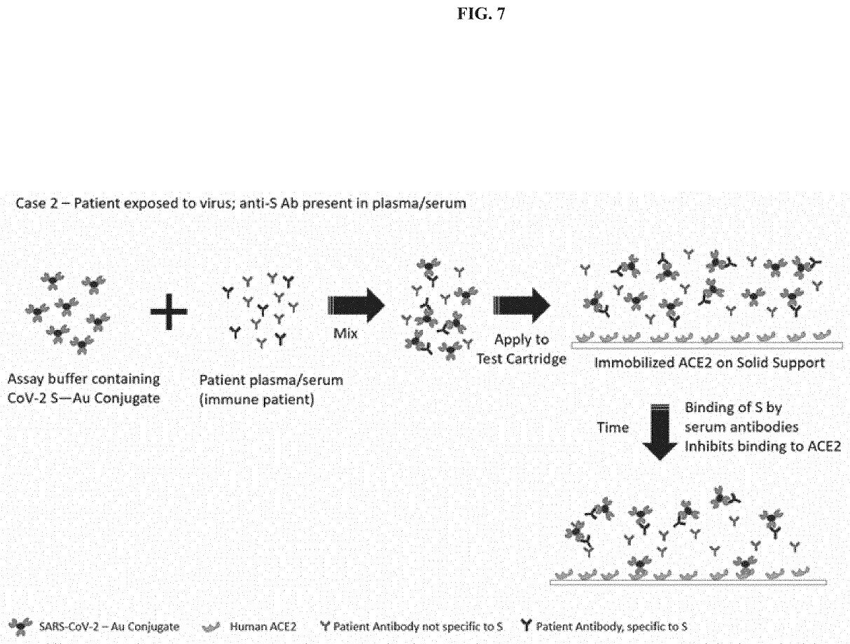

In some embodiments, the surface of the image is not captured. Referring to FIG. 7, a biological sample obtained from the subject exposed to a coronavirus is assayed using the assay assembly described here. A low number of complexes between the immobilized human ACE2 and the detectable peptide-conjugate is detectable by the human eye in the detection zone of the device.

In some embodiments, the assay assembly is a lateral flow assay (LFA). In some embodiments, the surface is a membrane comprising porous paper, a polymer structure, a sintered polymer, or a combination thereof. In some embodiments, the LFA assembly has one or more zones situated laterally, including a detectable zone. The detectable zone comprises at least a control region and a test region.

In some embodiments, the LFA assembly further comprises a sample receptor (e.g., a sample pad) configured to receive a biological sample. In some embodiments, the sample receptor comprises a filter designed to separate a component of the biological sample to be tested. For instance, if a biological sample were blood, the sample component would be blood serum. In some embodiments, the LFA assembly further comprises an absorbent pad.

In some instances, the target analyte moves without the assistance of external forces, e.g., by capillary action. In some instances, the target analyte moves with assistance of external forces, e.g., by facilitation of capillary action by movement of the lateral flow assembly (e.g., shaking, turning, centrifuging, applying an electrical field or magnetic field, applying a pump, applying a vacuum, or rocking).

Any suitable lateral flow test strip detection format known to those of skill in the art is contemplated for use in an assay assembly of the present disclosure. Lateral flow test strip detection formats are well known and have been described in the literature. Lateral flow test strip assay formats are generally described by, e.g., Sharma et al., (2015) Biosensors 5:577-601, incorporated by reference herein in its entirety. Detection of nucleic acids using lateral flow test strip sandwich assay formats is described by, e.g., U.S. Pat. No. 9,121,849, “Lateral Flow Assays,” incorporated by reference herein in its entirety. Detection of nucleic acids using lateral flow test strip competitive assay formats is described by, e.g., U.S. Pat. No. 9,423,399, “Lateral Flow Assays for Tagged Analytes,” incorporated by reference herein in its entirety.

Disclosed herein, in some embodiments, are signal detection devices, such as an imaging device. In some embodiments, the imaging device is a personal electronic device (e.g., smartphone or tablet). In some embodiments, the imaging device comprises a fluorescence reader, a colorimeter, or a sensor.

In some embodiments, the peptide-conjugate described herein comprises detection reagent or a label. Non-limiting examples of a detection reagent include a fluorophore, a chemical, a nanoparticle, an antibody, a peptide, and a nucleic acid probe. In some embodiments, the nanoparticle comprises a material selected from agarose, plastic, acrylic, or metal. In some embodiments, the nanoparticle is a microsphere. In some embodiments, the nanoparticle is magnetic. In some embodiments, the imaging device detects color, reflectance, fluorescence, bioluminescence, chemiluminescence, light, or an electrical signal.

In some embodiments, the LFA assembly is in a sandwich format, a competitive format, or a multiplex detection format. In a sandwich assay format, the detected signal is directly proportional to the amount of the target analyte present in the sample, so that increasing amounts of the target analyte lead to increasing signal intensity. In a competitive assay format, the detected signal has an inverse relationship with the amount of analyte present, and increasing amounts of analyte lead to decreasing signal intensity.

In a lateral flow sandwich format, also referred to as a “sandwich assay,” the biological sample (test sample) is applied to a sample receptor (“sample pad”) at a distal end of the LFA test strip. The biological sample flows through the test strip, from the sample pad to a conjugate pad located adjacent to, and downstream from, the sample pad. In some embodiments, the conjugate pad comprises a labeled capture molecule, e.g., an antibody or aptamer labeled with a dye, enzyme, or nanoparticle. A complex between the capture molecule and the target analyte is formed if the target analyte is present in the test sample. This complex then flows to a first test zone or sector (e.g., a test line) comprising an immobilized second capture molecule which is specific to the target analyte, thereby trapping any labeled capture molecule-target analyte complexes. In some embodiments, the intensity or magnitude of signal, e.g., color, fluorescence, reflectance, at the first test zone or sector is used to indicate the presence or absence, quantity, or presence and quantity of target analyte in the test sample. In some embodiments, the assay assembly comprises a second test zone or sector can comprise a third capture molecule that binds to excess labeled capture molecule. If the applied test sample comprises the target analyte, little or no excess labeled capture molecule will be present on the test strip following capture of the target analyte by the labeled capture molecule on the conjugate pad. Consequently, the second test zone or sector will not bind any labeled capture molecule, and little or no signal (e.g., color, fluorescence, reflectance) at the second test zone or sector is expected to be observed. The absence of signal at the second test zone or sector thus can provide assurance that signal observed in the first test zone or sector is due to the presence of the target analyte.

In some embodiments, the sandwich assay is configured to receive a biological sample disclosed herein and retain sample components (e.g., target analyte). In some embodiments, the sandwich assay is configured to receive a flow solution that flushes unwanted cellular components (other than the analyte) of the biological sample, leaving the target analyte behind. In some embodiments, the sandwich assay comprises a membrane that binds the target analyte to help retain the target analyte when the flow solution is applied. Non-limiting examples of a membrane the binds a target analyte includes chitosan modified nitrocellulose.

In some embodiments, the assay assembly comprises a sandwich assay. In some embodiments, the target analyte is an antibody specific to a pathogen of interest (e.g., SARS-CoV-2). In some cases, labeled capture molecule is a peptide derived from a spike protein from a coronavirus (e.g., SARS-CoV-2). In some embodiments, immobilized capture molecule is an antibody specific to the target analyte, such as an antibody or antigen-binding fragment. In some embodiments, signal is observed at the first test zone comprising color, fluorescence, or reflectance emitted from the labeled capture molecule. In some embodiments, the labeled capture molecule is a peptide is a peptide-conjugate comprising a peptide derived from a spike protein from a coronavirus, and a label comprising a nanoparticle (e.g., microsphere), an enzymatic label, or a fluorescent dye.

In a lateral flow competitive format, also referred to as a “competitive assay,” the test sample is applied to a sample pad at one end of the test strip, and the target analyte binds to a labeled capture molecule to form a complex between the target analyte and the labeled capture molecule in a conjugate pad downstream of the sample application pad. In the competitive format, the first test zone comprises an immobilized capture molecule specific to second capture molecule that is labeled. In the absence of the target analyte, the immobilized capture molecule and the labeled second capture molecule form a detectable complex. In the presence of the target analyte, fewer complexes between the capture molecule and the labeled second capture molecule form due to competition for binding to the labeled second capture molecule. In some embodiments, the intensity or magnitude of signal, e.g., color, fluorescence, reflectance, at the first test zone or sector is inversely proportionate to the presence or absence, quantity, or presence and quantity of the target analyte in the test sample.

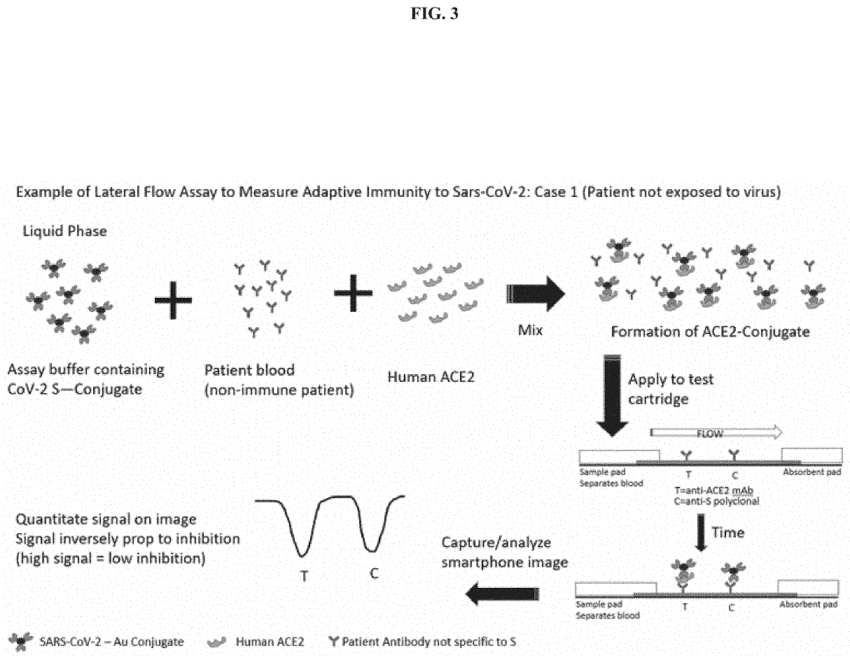

In some embodiments, the assay assembly comprises a competitive assay. Referring to FIG. 3, the labeled capture molecule comprises a peptide-conjugate comprising a detection agent (e.g., gold nanoparticle) and a peptide derived from the spike protein of a coronavirus. A fluid formulation comprising the peptide-conjugate is contacted with a biological sample from a patient and human ACE2, wherein the peptide derived from the spike protein of the coronavirus comprises a receptor binding domain (RBD) specific to the human ACE2. In this example, the target analyte is one or more antibodies against the spike protein of the corona virus. In this example the patient has not been exposed to the coronavirus, so a presence of the analyte is not expected to be detected.

The liquid formulation comprises at least one detectable complex comprising the peptide-conjugate RBD and the human ACE2, because the biological sample does not consist of antibodies against the RBD of the peptide. The liquid formulation comprising the peptide-conjugate, the biological sample, and the human ACE2 is applied to a solid surface at least partially enclosed in a housing (“test cartridge”). The liquid formulation is applied to the distal end of the solid surface at a sample pad, and flows unidirectionally over the solid surface towards the opposite distal end of the solid surface. In some embodiments, the biological sample is blood, and the sample pad separates serum from blood, permitting only the serum to flow across the solid surface. In some embodiments, the solid surface is a nitrocellulose membrane. In some embodiments, the first test zone comprises an immobilized antibody specific to the human ACE2. In some embodiments, the second test zone comprises an immobilized antibody specific to the RBD of the spike protein. In some embodiments, a high signal is observed at the first test zone, because there is an absence of target analyte in the biological sample. In some embodiments, a high signal is observed at the second test zone (positive control), for the same reason.

In alternative embodiments, the immobilized capture molecule in the first test zone is human ACE2 receptor, and the fluid formulation does not contain human ACE2. In this example, the liquid formulation comprises the peptide-conjugate and the biological sample. No complexes form between the peptide-conjugate in the absence of the target analyte (antibodies against the RBD of the spike protein). The liquid formulation is applied to the distal end of the solid surface at a sample pad, and flows unidirectionally over the solid surface towards the opposite distal end of the solid surface. Like above, in the first and second test zones, a high signal is observed in the absence of the test analyte.

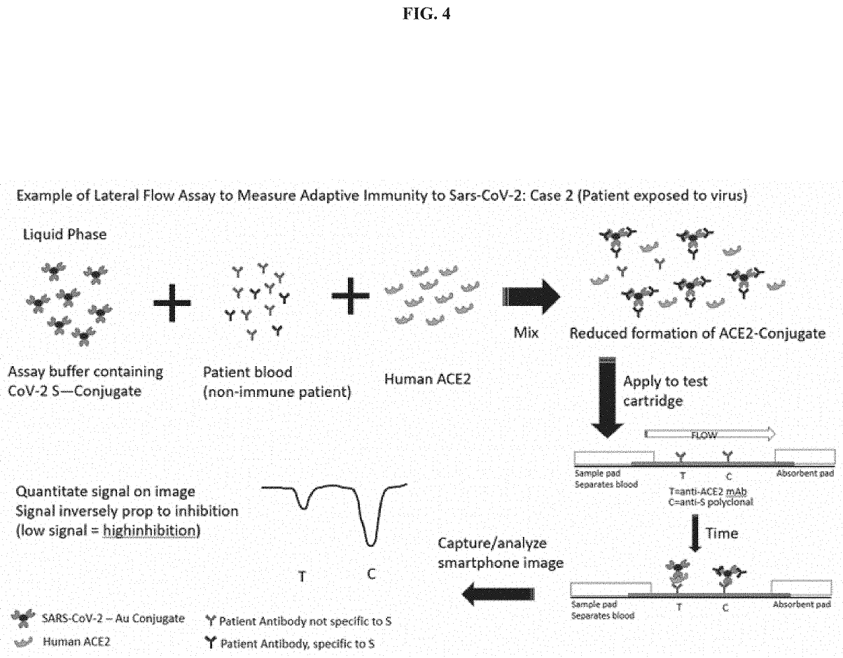

Referring to FIG. 4, the same competition assay is performed on biological sample it obtained from a subject that has been exposed to the coronavirus. In some embodiments, there is a presence of the target analyte in the biological sample. Therefore, a low signal is observed at the first and second test zones, which indicates a high amount of competitive binding between the target analyte and the peptide conjugate.

In a lateral flow test strip multiplex detection format, more than one target analyte is detected using the test strip through the use of additional test zones or sectors comprising, e.g., probes specific for each of the target analytes.

In some instances, the lateral flow device is a layered lateral flow device, comprising zones or sectors that are present in layers situated medially, e.g., above or below each other. In some instances, one or more zones or sectors are present in a given layer. In some instances, each zone or sector is present in an individual layer. In some instances, a layer comprises multiple zones or sectors. In some instances, the layers are laminated. In a layered lateral flow device, processes controlled by diffusion and directed by the concentration gradient are possible driving forces. For example, multilayer analytical elements for fluorometric assay or fluorometric quantitative analysis of an analyte contained in a sample liquid are described in EP0097952, “Multilayer analytical element,” incorporated by reference herein.

A lateral flow device can comprise one or more functional zones or sectors. In some embodiments, the test assembly comprises 1 to 20 functional zones or sectors. In some instances, the functional zones ore sectors comprise at least one sample purification zone or sector, at least one target analyte amplification zone or sector, at least one target analyte detection zone or sector, and at least one target analyte detection zone or sector.

In some embodiments, the assay assembly comprises a detection zone made up of at least the first and the second test zones (test, and control). In some embodiments, an image of the detection zone is captured with an imaging device. In some embodiments, the image is a video or still image. In some embodiments, the imaging device comprises a fluorescence reader, a colorimeter, or a sensor. In some embodiments, the imaging device is a personal electronic device, such as a smartphone.

In some embodiments, the assay assembly is a lab-on-chip system (e.g., Maverik®). In some embodiments, the solid surface comprises a silicon chip. In some embodiments, the imaging device comprises photonic biosensors that measure changes in refractive index caused by binding between the analyte and the peptide-conjugate to form complexes, as described in Iqbal M., et al. (2010) Label-Free Biosensor Arrays Based on Silicon Ring Resonators and High-Speed Optical Scanning Instrumentation. IEEE J Sel Quantum Elec 16.

### Testing Device Components

Disclosed herein, in some embodiments, are testing devices comprising a housing. In some embodiments, a testing device is at least partially enclosed by the housing. In some embodiments, the testing device is fully enclosed by the housing. In some embodiments, the house comprises a synthetic polymer material, such as plastic.

In some embodiments, the housing is configured to provide information to an imaging device described herein. Information can include, but is not limited to, information to normalize an image of the testing device and identifying information (e.g., barcode, RFID chip). In some embodiments, the identifying information comprises test parameters, test result interpretation instructions, expected values for imaging controls, and the like.

Disclosed herein, in some embodiments, are testing devices comprising a sample receptor. In some embodiments, the sample receptor is a sample receiver, a sample processor, a sample purifier, or a combination thereof. In some embodiments, the sample receptor is a sample receiver configured to receive and retain a biological sample obtained from a subject. In some embodiments, the sample receptor is a sample processor configured to remove a component of the sample or separate the sample into multiple fractions (e.g., blood cell fraction and plasma or serum).

Useful separation materials may include specific binding moieties that bind to or associate with the substance. Binding can be covalent or noncovalent. Any suitable binding moiety known in the art for removing a particular substance can be used. For example, antibodies and fragments thereof are commonly used for protein removal from samples. In some instances, a sample purifier disclosed herein comprises a binding moiety that binds a nucleic acid, protein, cell surface marker, or microvesicle surface marker in the biological sample. In some instances, the binding moiety comprises an antibody, antigen binding antibody fragment, a ligand, a receptor, a peptide, a small molecule, or a combination thereof.

The sample receptor is a sample purifier, configured to remove an unwanted substance or non-target component of a biological sample. Depending on the source of the biological sample, unwanted substances can include, but are not limited to, proteins (e.g., antibodies, hormones, enzymes, serum albumin, lipoproteins), free amino acids and other metabolites, microvesicles, nucleic acids, lipids, electrolytes, urea, urobilin, pharmaceutical drugs, mucous, bacteria, and other microorganisms, and combinations thereof. In some embodiments, the sample purifier separates components of a biological sample disclosed herein. In some embodiments, sample purifier disclosed herein removes one or more components of a sample that would inhibit, interfere with or otherwise be detrimental to the analyses of the target analyte. In some embodiments, the resulting modified sample is enriched for the target analyte. This can be considered indirect enrichment of target analytes. Alternatively or additionally, target analytes may be captured directly, which is considered direct enrichment of target analytes.

In some embodiments, sample purifiers disclosed herein comprise a filter. In some embodiments, sample purifiers disclosed herein comprise a membrane. Generally the filter or membrane is capable of separating or removing cells, cell particles, cell fragments, blood components other than cell-free nucleic acids, or a combination thereof, from the biological samples disclosed herein.

In some embodiments, the sample purifier facilitates separation of plasma or serum from cellular components of a blood sample. In some embodiments, the sample purifier facilitates separation of plasma or serum from cellular components of a blood sample before starting a molecular amplification reaction or a sequencing reaction. Plasma or serum separation can be achieved by several different methods such as centrifugation, sedimentation or filtration. In some embodiments, the sample purifier comprises a filter matrix for receiving whole blood, the filter matrix having a pore size that is prohibitive for cells to pass through, while plasma or serum can pass through the filter matrix uninhibited. In some embodiments, the filter matrix combines a large pore size at the top with a small pore size at the bottom of the filter, which leads to very gentle treatment of the cells preventing cell degradation or lysis, during the filtration process. This is advantageous because cell degradation or lysis would result in release of nucleic acids from blood cells or maternal cells that would contaminate target cell-free nucleic acids. Non-limiting examples of such filters include Pall Vivid™ GR membrane, Munktell Ahlstrom filter paper, and TeraPore filters.

In some embodiments a vertical filtration system is used to facilitate separation of plasma or serum from a cellular component of a blood sample. In this instance, the filtration is driven by capillary force to separate a component or fraction from a sample (e.g., plasma from blood). By way of non-limiting example, vertical filtration may comprise gravitation assisted plasma separation. A high-efficiency superhydrophobic plasma separator is described, e.g., by Liu et al., A High Efficiency Superhydrophobic Plasma Separation, Lab Chip 2015.

In some embodiments, the sample purifier comprises a lateral filter (e.g., sample does not move in a gravitational direction or the sample moves perpendicular to a gravitational direction). The sample purifier may comprise a vertical filter (e.g., sample moves in a gravitational direction). The sample purifier may comprise vertical filter and a lateral filter. The sample purifier may be configured to receive a sample or portion thereof with a vertical filter, followed by a lateral filter. The sample purifier may be configured to receive a sample or portion thereof with a lateral filter, followed by a vertical filter. In some embodiments, a vertical filter comprises a filter matrix. In some embodiments, the filter matrix of the vertical filter comprises a pore with a pore size that is prohibitive for cells to pass through, while plasma can pass the filter matrix uninhibited. In some embodiments, the filter matrix comprises a membrane that is especially suited for this application because it combines a large pore size at the top with a small pore size at the bottom of the filter, which leads to very gentle treatment of the cells preventing cell degradation during the filtration process.

In some embodiments, the filter comprises a material that moves, draws, pushes, or pulls the biological sample through the filter. In some embodiments, the material is a wicking material. Examples of appropriate materials used in the sample purifier to remove cells include, but are not limited to, polyvinylidene difluoride, polytetrafluoroethylene, acetylcellulose, nitrocellulose, polycarbonate, polyethylene terephthalate, polyethylene, polypropylene, glass fiber, borosilicate, vinyl chloride, or silver. In some embodiments, the separation material is a hydrophobic filter, for example a glass fiber filter, a composite filter, for example Cytosep (e.g., Ahlstrom Filtration or Pall Specialty Materials, Port Washington, N.Y.), or a hydrophilic filter, for example cellulose (e.g., Pall Specialty Materials). In some embodiments, whole blood can be fractionated into red blood cells, white blood cells and serum components for further processing according to the methods of the present disclosure using a commercially available kit (e.g., Arrayit Blood Card Serum Isolation Kit, Cat. ABCS, Arrayit Corporation, Sunnyvale, Calif.).

In some embodiments, the sample purifier comprises at least one filter or at least one membrane characterized by at least one pore size. In some embodiments, at least one pore size of at least one filter is about 0.05 microns to about 10 microns. In some embodiments, the pore size is about 0.05 microns to about 8 microns. In some embodiments, the pore size is about 0.05 microns to about 6 microns. In some embodiments, the pore size is about 0.05 microns to about 4 microns. In some embodiments, the pore size is about 0.05 microns to about 2 microns. In some embodiments, the pore size is about 0.05 microns to about 1 micron. In some embodiments, at least one pore size of at least one filter is about 0.1 microns to about 10 microns. In some embodiments, the pore size is about 0.1 microns to about 8 microns. In some embodiments, the pore size is about 0.1 microns to about 6 microns. In some embodiments, the pore size is about 0.1 microns to about 4 microns. In some embodiments, the pore size is about 0.1 microns to about 2 microns. In some embodiments, the pore size is about 0.1 microns to about 1 micron.

In some embodiments, the sample processor is configured to separate blood cells from whole blood. In some embodiments, the sample processor is configured to isolate plasma from whole blood. In some embodiments, the sample processor is configured to isolate serum from whole blood. In some embodiments, the sample processor is configured to isolate plasma or serum from less than 1 milliliter of whole blood. In some embodiments, the sample processor is configured to isolate plasma or serum from less than 1 milliliter of whole blood. In some embodiments, the sample processor is configured to isolate plasma or serum from less than 500 microliters (μL) of whole blood. In some embodiments, the sample processor is configured to isolate plasma or serum from less than 400 μL of whole blood. In some embodiments, the sample processor is configured to isolate plasma or serum from less than 300 μL of whole blood. In some embodiments, the sample processor is configured to isolate plasma or serum from less than 200 μL of whole blood. In some embodiments, the sample processor is configured to isolate plasma or serum from less than 150 μL of whole blood. In some embodiments, the sample processor is configured to isolate plasma or serum from less than 100 μL of whole blood.

Disclosed herein, in some embodiments, are devices comprising a detection zone. In some embodiments, the detection zone comprises a test region and a control region. In some embodiments, the imaging device captures an image of the detection zone. In some embodiments, the control region is a positive control. In some embodiments, the control region is a negative control. In some embodiments, the control region comprises a positive and a negative control.

### II. Systems

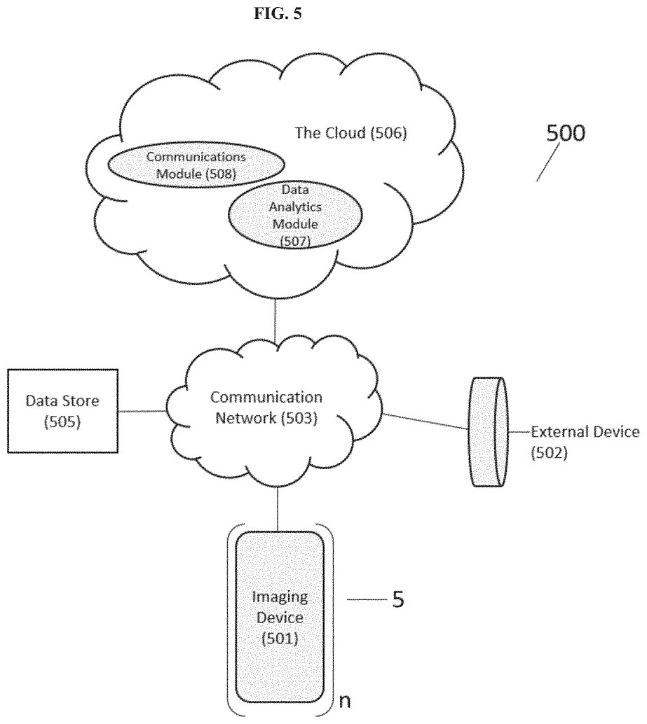

Disclosed herein, in some embodiments, are systems. Referring to FIG. 5, the system 500 comprises, in some embodiments, a testing device, an imaging device 501, and a computing device, for determining whether a subject is immune to an infection by a pathogen of interest (e.g., SARS-CoV-2). The imaging device and/or the computing device are configured to receive and analyze data generated by the testing device and/or one or more external devices, to provide a result to the subject. The result is provided to the subject via a graphical user interface (GUI) by the imaging device and/or computing device using an application (web application or mobile application). Systems further comprise one or more data store for storing and retrieving the data.

Disclosed herein, in some embodiments, are data analyzed by one or more components of the system described herein. In some embodiments, the data are structured or unstructured. In some embodiments, the data are generated by the imaging device when an image is captured of the detection zone of the testing devices described herein. In some embodiments, the data are external data generated from an external device. In some embodiments, the external device comprises a diagnostic device, a prognostic device, or a health or fitness tracking device. In some embodiments, the external data comprise body temperature, heart rate variability, resting heart rate, sleep quality, or sleep quantity, or a combination thereof.

Provided here, in some embodiments, are imaging devices for capturing an image of the detection zone of the testing devices described herein. In some embodiments, the imaging device is a camera. In some embodiments, the imaging device is a computing device with a camera, such as a smartphone, laptop, or tablet. In some embodiments, the imaging device is not a computing device, but is in communication with the computing device via a communication network. In some embodiments, the communication network is wireless, such as wireless Internet or Bluetooth.

Referring to FIG. 5, the system 500 comprises an imaging device 501, an external device 502, a data store 505, all in communication via a communication network 503, equipped with cloud-based computing executed by the data analytics module 507 and communications module 508. In this example, the imaging device transmits data from an image captured of the detection zone of the testing device described herein, via the communication network, to the data analytics module 507 in the cloud 506 to be analyzed. The data analytics module 507 transmits the result to the communications module 508 to be packaged for display to the user. In this example, the result is displayed on the imaging device 501, which is a personal electronic device belonging to the user (i.e., the subject in this example). The data store 505 is a remote server in this example. Alternatively, the data store is a cloud-based data store.

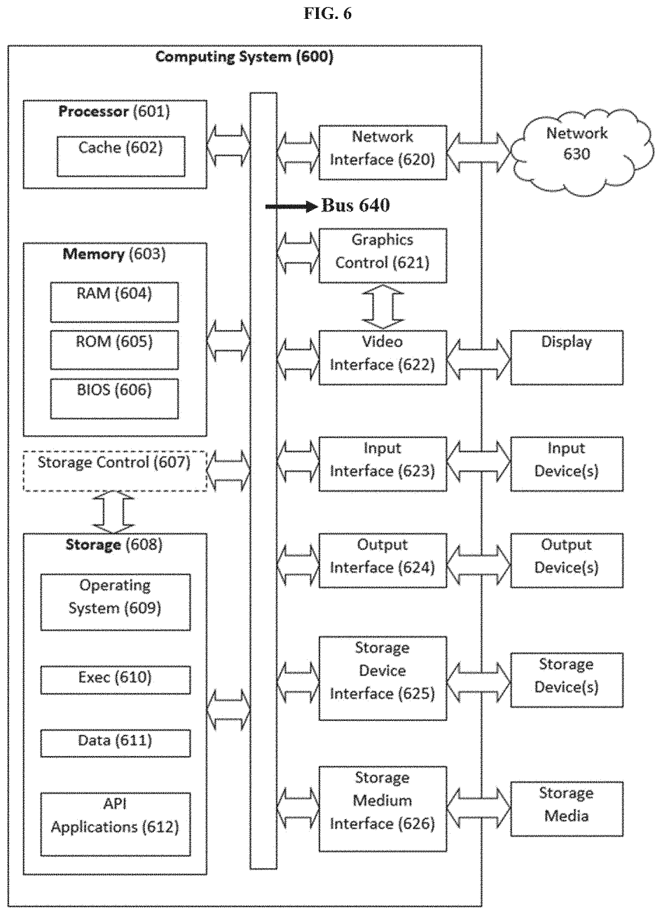

Also provided, in some embodiments, are computing devices comprising a computing system configured to analyze data described herein to provide a result. Referring to FIG. 6, a block diagram is shown depicting an exemplary computing device that includes a computing system 600 (e.g., a processing or computing system) within which a set of instructions can execute for causing a device to perform or execute any one or more of the aspects and/or methodologies for static code scheduling of the present disclosure. The components in FIG. 6 are examples only and do not limit the scope of use or functionality of any hardware, software, embedded logic component, or a combination of two or more such components implementing particular embodiments.

Computer system 600 can include one or more processors 601, a memory 603, and a storage 608 that communicate with each other, and with other components, via a bus 640. The bus 640 can also link a display 632, one or more input devices 633 (which may, for example, include a keypad, a keyboard, a mouse, a stylus, etc.), one or more output devices 634, one or more storage devices 635, and various tangible storage media 636. All of these elements can interface directly or via one or more interfaces or adaptors to the bus 640. For instance, the various tangible storage media 636 can interface with the bus 640 via storage medium interface 626. Computer system 600 can have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 600 includes one or more processor(s) 601 (e.g., central processing units (CPUs) or general purpose graphics processing units (GPGPUs)) that carry out functions. Processor(s) 601 optionally contains a cache memory unit 602 for temporary local storage of instructions, data, or computer addresses. Processor(s) 601 are configured to assist in execution of computer readable instructions. Computer system 600 can provide functionality for the components depicted in FIG. 6 as a result of the processor(s) 601 executing non-transitory, processor-executable instructions embodied in one or more tangible computer-readable storage media, such as memory 603, storage 608, storage devices 635, and/or storage medium 636. The computer-readable media can store software that implements particular embodiments, and processor(s) 601 can execute the software. Memory 603 can read the software from one or more other computer-readable media (such as mass storage device(s) 635, 636) or from one or more other sources through a suitable interface, such as network interface 620. The software can cause processor(s) 601 to carry out one or more processes or one or more steps of one or more processes described or illustrated herein. Carrying out such processes or steps can include defining data structures stored in memory 603 and modifying the data structures as directed by the software.

The memory 603 can include various components (e.g., machine readable media) including, but not limited to, a random access memory component (e.g., RAM 604) (e.g., static RAM (SRAM), dynamic RAM (DRAM), ferroelectric random access memory (FRAM), phase-change random access memory (PRAM), etc.), a read-only memory component (e.g., ROM 605), and any combinations thereof. ROM 605 can act to communicate data and instructions unidirectionally to processor(s) 601, and RAM 604 can act to communicate data and instructions bidirectionally with processor(s) 601. ROM 605 and RAM 604 can include any suitable tangible computer-readable media described below. In one example, a basic input/output system 606 (BIOS), including basic routines that help to transfer information between elements within computer system 600, such as during start-up, can be stored in the memory 603.

Fixed storage 608 is connected bidirectionally to processor(s) 601, optionally through storage control unit 607. Fixed storage 608 provides additional data storage capacity and can also include any suitable tangible computer-readable media described herein. Storage 608 can be used to store operating system 609, executable(s) 610, data 611, applications 612 (application programs), and the like. Storage 608 can also include an optical disk drive, a solid-state memory device (e.g., flash-based systems), or a combination of any of the above. Information in storage 608 may, in appropriate cases, be incorporated as virtual memory in memory 603.

In one example, storage device(s) 635 can be removably interfaced with computer system 600 (e.g., via an external port connector (not shown)) via a storage device interface 625. Particularly, storage device(s) 635 and an associated machine-readable medium can provide non-volatile and/or volatile storage of machine-readable instructions, data structures, program modules, and/or other data for the computer system 600. In one example, software can reside, completely or partially, within a machine-readable medium on storage device(s) 635. In another example, software can reside, completely or partially, within processor(s) 601.

Bus 640 connects a wide variety of subsystems. Herein, reference to a bus can encompass one or more digital signal lines serving a common function, where appropriate. Bus 640 can be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 600 can also include an input device 633. In one example, a user of computer system 600 can enter commands and/or other information into computer system 600 via input device(s) 633. Examples of an input device(s) 633 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 633 can be interfaced to bus 640 via any of a variety of input interfaces 623 (e.g., input interface 623) including, but not limited to, serial, parallel, game port, USB, FIREWIRE, THUNDERBOLT, or any combination of the above.

In particular embodiments, when computer system 600 is connected to network 630, computer system 600 can communicate with other devices, specifically mobile devices and enterprise systems, distributed computing systems, cloud storage systems, cloud computing systems, and the like, connected to network 630. Communications to and from computer system 600 can be sent through network interface 620. For example, network interface 620 can receive incoming communications (such as requests or responses from other devices) in the form of one or more packets (such as Internet Protocol (IP) packets) from network 630, and computer system 600 can store the incoming communications in memory 603 for processing. Computer system 600 can similarly store outgoing communications (such as requests or responses to other devices) in the form of one or more packets in memory 603 and communicated to network 630 from network interface 620. Processor(s) 601 can access these communication packets stored in memory 603 for processing.

Examples of the network interface 620 include, but are not limited to, a network interface card, a modem, and any combination thereof. Examples of a network 630 or network segment 630 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 630, can employ a wired and/or a wireless mode of communication. In general, any network topology can be used.

Information and data can be displayed through a display 632. Examples of a display 632 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 632 can interface to the processor(s) 601, memory 603, and fixed storage 608, as well as other devices, such as input device(s) 633, via the bus 640. The display 632 is linked to the bus 640 via a video interface 622, and transport of data between the display 632 and the bus 640 can be controlled via the graphics control 621. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 632, computer system 600 can include one or more other peripheral output devices 634 including, but not limited to, an audio speaker, a printer, a storage device, and any combinations thereof. Such peripheral output devices can be connected to the bus 640 via an output interface 624. Examples of an output interface 624 include, but are not limited to, a serial port, a parallel connection, a USB port, a FIREWIRE port, a THUNDERBOLT port, and any combinations thereof.

In addition or as an alternative, computer system 600 can provide functionality as a result of logic hardwired or otherwise embodied in a circuit, which can operate in place of or together with software to execute one or more processes or one or more steps of one or more processes described or illustrated herein. Reference to software in this disclosure can encompass logic, and reference to logic can encompass software. Moreover, reference to a computer-readable medium can encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein can be embodied directly in hardware, in a software module executed by one or more processor(s), or in a combination of the two. A software module can reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, smart phone, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art. In some embodiments, the smart phone is an Apple iPhone or an android device (e.g., Samsung Galaxy).

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

Disclosed herein are computing systems comprising a data processor. In some embodiments, the data processor is a mobile processor. In some embodiments, the data processor is configured to receive data from an imaging device, an external device, or a data store, or a combination thereof. In some embodiments, data processor analyzes the data to provide a result. In some embodiments, the imaging device and the data processor are housed in the same device, such as a smartphone.

In some embodiments, the data processor is configured to provide a computer program or application, comprising a data analytics module. In some embodiments, the application is a web application. In some embodiments, the application is a mobile application. In some embodiments, the data analytics module is configured to receive data from an imaging device and analyze the data to provide a result. In some embodiments, the data analytics module is configured to receive external data from an external device. In some embodiments, the external data comprises body temperature, heart rate variability, resting heart rate, sleep quality, or sleep quantity, or a combination thereof. In some embodiments, external device comprises a diagnostic device, a prognostic device, or a health or fitness tracking device. In some embodiments, the health tracking device is the Aurora®, Fitbit®, or Apple Watch. In some embodiments, systems comprise multiple external devices. In some embodiments, the data analytics module is configured to analyze the external data to provide a result. In some embodiments, the data analytics module is configured to analyze the external data and the data received from the imaging device to provide a result.

In some embodiments, the mobile application comprises a communication module. In some embodiments, the communication module is configured to communicate the result to the subject. In some embodiments, the communication module is configured to display the result to the subject via a graphical user interface (GUI) of an electronic device. In some embodiments, the electronic device is the imaging device described herein, the computing device described herein, or a combination thereof. In some embodiments, the electronic device is a smartphone, such as those described herein.

### Computer Program

Disclosed herein, in some embodiments, the data processor is configured to run a computer program. A computer program includes a sequence of instructions, executable by one or more processor(s) of the computing device's CPU, written to perform a specified task. Computer readable instructions can be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program can be written in various versions of various languages.

The functionality of the computer readable instructions can be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media

### Web Application

Disclosed here, in some embodiments, are data processors comprising one or more web applications. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application can be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®

### Mobile Application

Also disclosed herein, in some embodiments, are data processors comprising one or more mobile applications. In some embodiments, the mobile application is provided to a mobile digital processing device at the time it is manufactured. In some embodiments, the mobile application is provided to a mobile digital processing device via the computer network described herein. Mobile applications disclosed herein can be configured to locate, encrypt, index, and/or access information. Mobile applications disclosed herein can be configured to acquire, encrypt, create, manipulate, index, and peruse data.

A mobile application is created by suitable techniques using hardware, languages, and development environments known to the art. Suitable programming languages include, by way of non-limiting examples, C, C++, C #, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, and Samsung® Apps.

### Standalone Application

Disclosed here, in some embodiments, are data processors comprising one or more standalone applications. A standalone application is an independent computer process; not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

### Software Modules

Disclosed herein, in some embodiments, the data processor comprises a computer program configured with one or more software modules. In some embodiments, the one or more software module is a data analytics module. In some embodiments, the one or more software module is a communication module.

In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location

In some embodiments, the computing system described herein comprise a data analytics module. In some embodiments, the data analytics module is configured to run one or more algorithms. In some embodiments, the one or more algorithms comprise a machine learning algorithm. The machine learning algorithm can be capable of supervised learning, unsupervised learning, reinforcement learning, semi-supervised learning, self-supervised learning, multi-instance learning, inductive learning, deductive inference, transduction learning, multi-task learning, active learning, online learning, transfer learn, or ensemble learning. In some embodiments, the data analytics module is configured with artificial intelligence (AI), such as a limited memory AI.

In some embodiments, the data analytics module receives data from the testing device, and optionally, external data from one or more external devices, and analyzes the data to provide a result. Referring to analysis of the data received from the testing device, the data analytics module normalizes the result by subtracting background signal intensity of the testing device (e.g., background plasma concentration, non-specific binding to the solid surface). Referring to external data, the data analytics module identifies whether external data are symptoms to an acute infection by a pathogen.

In some embodiments, the result is either positive or negative. In some embodiments, the positive indicates an acute infection and negative indicates a lack of an acute infection. In some embodiments, the positive result indicates that the subject immune to a future infection by a pathogen of interest. In some embodiments, the data analytics module transmits the result to a communications module for display to the subject.

In some embodiments, the communication module is configured to display one or more results to the subject via a graphical user interface (GUI).

### Data Store

Disclosed herein, in some embodiments, are systems comprising one or more data stores. In some embodiments, the data store is a database suitable for the storage and retrieval of data. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In a particular embodiment, a database is a distributed database. In other embodiments, a database is based on one or more local computer storage devices, such as a smartphone.

### Graphical User Interface

Also disclosed herein, in some embodiments, are graphical user interfaces (GUI) configured to display a result to a user. In some embodiments, the user is the subject. In some embodiments, the GUI comprises one or more dashboards of an application (e.g., web application or mobile application). In some embodiments, the dashboard comprises relevant health information to a subject. In some embodiments, the GUI is a part of personal electronic device, such as a smartphone or tablet, belonging to the user.

### Web Portal

Disclosed herein, in some embodiments, is a web portal providing a single access point for multiple users to access information about the immune status of a subject. In some embodiments, a web portal provides access to a subject, a subject's doctor, or a healthcare worker responding to an urgent public health crisis. In some embodiments, the portal provides a single access point for a population of individuals, wherein the data is anonymized. In this example, the web portal provides access to policy makers, health care professionals, governmental organization, and non-governmental organizations responding to a public health crisis. Among other information, the portal can indicate one or more geographical locations comprises of subject either acutely infected by a pathogen of interest, or immune to the pathogen of interest.

### III. Methods

Disclosed herein, in some embodiments, are methods of measuring a target analyte in biological sample obtained from subject. In some embodiments, methods comprise utilizing the testing devices described herein. In some embodiments, methods further comprise analyzing data generated by the testing device disclosed herein, and providing a result to a user of an electronic device. In some embodiments, providing the result comprises displaying the result on a GUI of the electronic device. In some embodiments, the analyzing and displaying is performed by a single computing device (e.g., smartphone, tablet). In some embodiments, analyzing and displaying is performed at the point of need (e.g., at the time and space that the analyte is detected in the biological sample using the testing device described herein).

In some embodiments, methods further comprise determining whether the subject is immune to an infection by a pathogen of interest (e.g., SARS-CoV-2). In some embodiments, methods further comprise determining whether a vaccine administered to the subject is effective to immunize the subject against the pathogen of interest. In some embodiments, the methods further comprise determining whether the biological sample is safe for transfusion into another subject (e.g., blood, or blood plasma transfusion). In some embodiments, the methods further comprise identifying the subject as having an acute infection by the pathogen of interest.

Disclosed herein, in some embodiments, are methods of identifying adaptive immunity to a pathogen in a subject, the method comprising: (a) obtaining a biological sample from the subject; (b) measuring a presence, an absence, or a level of a complex between an analyte in the biological sample by detecting a number of complexes between a detectable peptide-conjugate and the analyte, wherein if the number of the complexes is high relative to an index or a control, then the subject is immune to an infection by the pathogen, and if the number of the complexes is low relative to an index or a control, then identifying the subject as not being immune to the infection by the pathogen. In some embodiments, the methods described herein are performed using the testing device of the present disclosure.

In some embodiments, the pathogen is a virus, a bacterium, a fungus, or a parasite. In some embodiments, the virus is a DNA virus or an RNA virus. In some embodiments, the virus is a single stranded virus, or a double stranded virus. In some embodiments, the virus is a plus strand or a minus strand DNA or RNA virus. In some embodiments, the virus replicates through reverse transcription of an RNA intermediate. In some embodiments, the virus is a coronavirus. In some embodiments, the coronavirus is a severe acute respiratory syndrome coronavirus (SARS-CoV). In some embodiments, the SARS-CoV is SARS-CoV-2. In some embodiments, the coronavirus is Middle East Respiratory Syndrome coronavirus (MERS-CoV). In some embodiments, the coronavirus is an alpha coronavirus (e.g., 229E, NL63). In some embodiments, the coronavirus is a beta coronavirus (e.g., OC43, HKU1).

In some embodiments, the subject is a human subject. In subject embodiments, the subject is pediatric (e.g., age 0-18). In some embodiments, the subject is not pediatric. In some embodiments, the subject is female or male. In some embodiments, the subject has been exposed to the pathogen. In some embodiments, the subject has not been exposed to the pathogen. In some embodiments, the subject exhibits one or more symptoms comprising a cough, fever, tiredness, or difficulty breathing. In some embodiments, the subject has an underlying health problem comprising high blood pressure, a heart problem, diabetes, immunodeficiency, autoimmune disease. In some embodiments, the subject is immunocompromised.

In some embodiments, obtaining can be direct or indirect. Indirectly obtaining a biological sample from a subject may include receiving it from a laboratory or processing/storage facility by mail, or otherwise. Directly obtaining the biological sample from the subject can be performed by a doctor or the subject at the point of need. In some embodiments, the biological sample is a biological fluid. In some embodiments, the biological sample is a swab sample (e.g., buccal swab, nasopharyngeal swab). In some embodiments, methods disclosed herein comprise obtaining whole blood, plasma, serum, urine, saliva, fecal matter, or interstitial fluid. In some embodiments, the blood is capillary blood. In some embodiments, the blood is not venous blood (e.g., from a phlebotomy). In some instances, methods disclosed herein comprise obtaining a blood sample by administering a finger prick.

In some embodiments, the analyte comprises an antibody against an antigenic peptide derived from the pathogen. As a non-limiting example, the analyte may be an antibody against apportion of the spike protein derived from a coronavirus (e.g., SARS-CoV-2). In some embodiments, the analyte is the activity of the antibody. In some embodiments, the activity is blocking binding between the spike protein of a coronavirus and its cognate receptor (e.g., ACE2). In some embodiments, the analyte is a complex comprising the spike protein bound to the antibody at the receptor binding region of the spike protein. In some embodiments, the antibody belongs to an immunoglobulin class comprising immunoglobulin M, immunoglobulin G, immunoglobulin A, immunoglobulin E, or immunoglobulin D.

In some embodiments, the detectable peptide-conjugate is the peptide-conjugate described herein comprising a detection agent and a peptide. In some embodiments, the peptide is an antigenic peptide. In some embodiments, the analyte (e.g., antibody) is specific to at least a portion of the peptide. In some embodiments, the peptide comprises at last a portion of the spike glycoprotein of a coronavirus (e.g., SARS-CoV-2) described herein. In some embodiments, the peptide is a receptor to the antigenic peptide. In some embodiments, the receptor comprises ACE2 receptor described herein.

In some embodiments, measuring comprises performing an assay on the biological sample to detect a number of complexes formed between the analyte and the peptide-conjugate. In some embodiments, the assay comprises an assay assembly described herein. In some embodiments, the assay is a lateral flow assay. In some embodiments, the lateral flow assay is a competition assay.

In some embodiments, the index or the control is derived from a patient that has not been exposed to the pathogen (negative control). In some embodiments, the index or control is from a patient that has been exposed to the pathogen (positive control).

Methods described herein, in some embodiments, do not consist of utilizing a cell culture, such as an immortalized cell line expressing human ACE2. In some embodiments, methods do not consist of handling or administering to a cell or cell line a purified or isolated pathogen, such as a live virus of pseudovirus.

Methods disclosed herein further comprise: (a) capturing an image of a detection zone of the testing device; and (b) analyzing data from the image using one or more computer programs. In some embodiments, the one or more computer programs is run on a computing device described here. In some embodiments, capturing is performed with an imaging device described herein. In some cases, capturing and analyzing are performed by a single personal electronic device, such as a smartphone, tablet, or laptop computer. In some embodiments, capturing and analyzing are performed at the point of need (e.g., same time and place as performing the assay with the testing device).

In some embodiments, capturing comprises taking a still photograph of a liquid phase, such as a liquid composition disclosed herein. In some embodiments, capturing comprises taking a still photograph of the solid surface of the assay assembly described herein. In some embodiments, capturing comprises taking a video of the solid surface of the assay assembly.

In some embodiments, analyzing comprises detecting binding between the analyte (antibody against an antigenic peptide from the pathogen) and peptide-conjugate. In some embodiments, analyzing comprises subtracting a background signal, thereby increasing the signal to noise ratio. In some embodiments, analyzing is performed by a data analytics module of an application or computer program of the computing device. In some embodiments, the analyzing by the data analytics module comprises performing machine learning.

### IV. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The term “analyte” refers to a substance whose chemical constituents or activity is measured. In some embodiments, the analyte comprises an activity of a neutralizing antibody. In some embodiments, the activity is blocking binding between a pathogen of interest (e.g., SARS-CoV-2) and a cognate receptor (e.g., ACE2). In some embodiments, the analyte is a complex comprising the spike protein bound to the antibody at the receptor binding region of the spike protein.

The term “cloud” refers to shared or sharable storage of electronic data. The cloud may be used for archiving electronic data, sharing electronic data, and analyzing electronic data.

As used herein, the terms, “clinic,” “clinical setting,” “laboratory” or “laboratory setting” refer to a hospital, a clinic, a pharmacy, a research institution, a pathology laboratory, a or other commercial business setting where trained personnel are employed to process and/or analyze biological and/or environmental samples. These terms are contrasted with point of care, a remote location, a home, a school, and otherwise non-business, non-institutional setting.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### V. Examples

The following examples are included for illustrative purposes only and are not intended to limit the scope of the invention.

### Example 1: Detecting Adaptive Immunity in a Healthcare Worker

A healthcare worker, responding to an urgent public health crisis, needs to know whether she is immune to an infection of a pathogen of interest so that she may serve others in the community at risk for infection. In this example, the pathogen of interest is SARS-CoV-2, and the healthcare worker has been exposed to SARS-CoV-2. Optionally, the healthcare worker has recovered from coronavirus disease of 2019 (COVID-19).

While at home, the healthcare worker utilizes the point of need testing device described herein to obtain capillary blood by pricking her finger. She applies the capillary blood to the sample receptor component of the testing device, where it separates the blood serum (containing the analyte of interest) and other blood components. The serum flows downstream from the sample receptor to a test zone of the testing device, where the serum is mixed with a fluid composition comprising a peptide-conjugate. In this example, the peptide comprises an amino acid sequence that is at least 95% identical to any one of SEQ ID NOS: 2-4. The peptide conjugate comprises a detectable moiety comprising a gold microsphere. Within 10-20 minutes a signal develops in the detectable zone of the testing device.

The healthcare worker takes a picture of the detectable zone using her smartphone, which is equipped with a mobile application that will interpret the results from the testing device. The mobile application displays the result via the graphical user interface of the smartphone and indicates to the healthcare worker that she is immune to the pathogen. She returns to work immediately.

### Example 2: Detecting Adaptive Immunity to Pathogen in Blood or Blood Plasma

A donation of blood or blood plasma is tested for a presence of neutralizing antibodies that functionally block binding between a pathogen of interest and an pathogen recognition receptor. In this example, the test is performed at the point of need (e.g., a blood bank) by a technician. In this example, the pathogen of interest is SARS-CoV-2.

At the blood donation site, the technician utilizes the point of need testing device described herein to test a sample of the blood. The technician applies the blood to the sample receptor component of the testing device, where it separates the blood serum (containing the analyte of interest) and other blood components. The serum flows downstream from the sample receptor to a test zone of the testing device, where the serum is mixed with a fluid composition comprising the peptide-conjugate from Example 1. Within 10-20 minutes a signal develops in the detectable zone of the testing device.

The technician takes a picture of the detectable zone using a tablet, which is equipped with a mobile application that will interpret the results from the testing device. The mobile application displays the result via the graphical user interface of the tablet and indicates to the technician that the blood sample contains neutralizing antibodies that block binding between the spike protein of SARS-CoV-2 and human ACE2.

The technician sends the blood sample to a research laboratory to determine whether the serum isolated from this blood (containing the neutralizing antibodies) is suitable for use in convalescent plasma therapy.

### Example 3. Vaccine Development Tool and Methods of Use

A pharmaceutical company is developing a vaccine to a pathogen of interest. In this example, the pathogen of interest is SARS-CoV-2. The pharmaceutical company wants to know whether the vaccine induces the production of antibodies which block the interaction between the spike protein of SARS—Co-V-2 and the human ACE2 receptor, that mediates infection in vivo. The pharmaceutical company utilizes the testing device described herein to test the vaccine by testing a biological sample from an animal (e.g., mammal) inoculated with the vaccine.

A biological sample is obtained from a mammal that has not been exposed to SARS—Co-V-2, and has been administered the vaccine. A researcher at the pharmaceutical company applies the biological sample to the sample receptor component of the testing device, where it separates the blood plasma/serum (containing the analyte of interest) and other blood components. The serum flows downstream from the sample receptor to a test zone of the testing device, where the serum is mixed with a fluid composition comprising the peptide-conjugate from Example 1. Within 10-20 minutes a signal develops in the detectable zone of the testing device.

The researcher takes an image of the detectable zone using an imaging device, which is equipped with an application that will interpret the results from the testing device. The application displays the result via the graphical user interface of the tablet and indicates to the researcher that biological sample contains antibodies that block the interaction between the spike protein of SARS—Co-V-2 and the human ACE2 receptor, which means that vaccine was effective. The vaccine moves on for further research and development.

### Example 4. Identifying Herd Immunity Using Artificial Intelligence

A governmental agency, responding to a global pandemic, is monitoring a population of citizens to determine whether a threshold adaptive immunity neutralizing a pathogen of interest is present in the population, such that incidences of infection are drastically reduced (also referred to as “herd immunity”). The agency provides citizens testing devices, such as those described herein; and a free mobile App that can be downloaded to their mobile device. Citizens in a given population utilize the testing device, take a picture of the testing zone of the testing device using their mobile device.

The data is anonymized and uploaded into the cloud. Data includes GPS data from the mobile devices and the data from the picture that was taken for each citizen. The data is analyzed by cloud-computing by machine learning, and results are accessible to the agency via a web-portal. The results are used to identify populations of citizens that are immune from SARS-CoV-2. Geofencing is used to create geographical boundaries around where those populations reside. When a threshold number of citizens with adaptive immunity neutralizing SARS-CoV-2 is detected conferring herd immunity.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

SEQUENCES

| | |
|---|---|
| # | SEQUENCE |

>sp|Q9BYF1|ACE2_HUMAN Angiotensin-converting enzyme 2 OS = Homo sapiens
OX = 9606 GN = ACE2 PE = 1 SV = 2

| | |
|---|---|
| SEQ ID | MSSSSWLLLSLVAVTAAQSTIEEQAKTFLDKFNHEAEDLFYQSSLASWNYNTN |
| NO: 1 | ITEENVQNMNNAGDKWSAFLKEQSTLAQMYPLQEIQNLTVKLQLQALQQNGS |
| | SVLSEDKSKRLNTILNTMSTIYSTGKVCNPDNPQECLLLEPGLNEIMANSLDYN |
| | ERLWAWESWRSEVGKQLRPLYEEYVVLKNEMARANHYEDYGDYWRGDYEV |
| | NGVDGYDYSRGQLIEDVEHTFEEIKPLYEHLHAYVRAKLMNAYPSYISPIGCLP |
| | AHLLGDMWGRFWTNLYSLTVPFGQKPNIDVTDAMVDQ |
| | AWDAQRIFKEAEKFFVSVGLPNMTQGFWENSMLTDPGNVQKAVCHPTAWDL |
| | GKGDFRILMCTKVTMDDFLTAHHEMGHIQYDMAYAAQPFLLRNGANEGFHE |
| | AVGEIMSLSAATPKHLKSIGLLSPDFQEDNETEINFLLKQALTIVGTLPFTYMLE |
| | KWRWMVFKGEIPKDQWMKKWWEMKREIVGVVEPVPHDETYCDPASLFHVS |
| | NDYSFIRYYTRTLYQFQFQEALCQAAKHEGPLHKCDISNSTEAGQKLFNMLRL |
| | GKSEPWTLALENVVGAKNMNVRPLLNYFEPLFTWLKDQNK |
| | NSFVGWSTDWSPYADQSIKVRISLKSALGDKAYEWNDNEMYLFRSSVAYAM |
| | RQYFLKVKNQMILFGEEDVRVANLKPRISFNFFVTAPKNVSDIIPRTEVEKAIR |
| | MSRSRINDAFRLNDNSLEFLGIQPTLGPPNQPPVSIWLIVFGVVMGVIVVGIVILI |
| | FTGIRDRKKKNKARSGENPYASIDISKGENNPGFQNTDDVQTSF |

>sp|P59594|SPIKE_CVHSA Spike glycoprotein OS = Human SARS coronavirus
OX = 694009 GN = S PE = 1 SV = 1

| | |
|---|---|
| SEQ ID | MFIFLLFLTLTSGSDLDRCTTFDDVQAPNYTQHTSSMRGVYYPDEIFRSDTLYL |
| NO: 2 | TQDLFLPFYSNVTGFHTINHTFGNPVIPFKDGIYFAATEKSNVVRGWVFGSTMN |
| | NKSQSVIIINNSTNVVIRACNFELCDNPFFAVSKPMGTQTHTMIFDNAFNCTFEY |
| | ISDAFSLDVSEKSGNFKHLREFVFKNKDGFLYVYKGYQPIDVVRDLPSGFNTLK |
| | PIFKLPLGINITNFRAILTAFSPAQDIWGTSAAAYFVGYLKPTTFMLKYDENGTI |
| | TDAVDCSQNPLAELKCSVKSFEIDKGIYQTSNFRVVPSGDVVRFPNITNLCPFG |
| | EVFNATKFPSVYAWERKKISNCVADYSVLYNSTFFSTFKCYGVSATKLNDLCF |
| | SNVYADSFVVKGDDVRQIAPGQTGVIADYNYKLPDDFMGCVLAWNTRNIDAT |
| | STGNYNYKYRYLRHGKLRPFERDISNVPFSPDGKPCTPPALNCYWPLNDYGFY |
| | TTTGIGYQPYRVVVLSFELLNAPATVCGPKLSTDLIKNQCVNFNFNGLTGTGVL |
| | TPSSKRFQPFQQFGRDVSDFTDSVRDPKTSEILDISPCSFGGVSVITPGTNASSEV |
| | AVLYQDVNCTDVSTAIHADQLTPAWRIYSTGNNVFQTQAGCLIGAEHVDTSY |
| | ECDIPIGAGICASYHTVSLLRSTSQKSIVAYTMSLGADSSIAYSNNTIAIPTNFSISI |
| | TTEVMPVSMAKTSVDCNMYICGDSTECANLLLQYGSFCTQLNRALSGIAAEQ |
| | DRNTREVFAQVKQMYKTPTLKYFG |
| | GFNFSQILPDPLKPTKRSFIEDLLFNKVTLADAGFMKQYGECLGDINARDLICA |
| | QKFNGLTVLPPLLTDDMIAAYTAALVSGTATAGWTFGAGAALQIPFAMQMAY |
| | RFNGIGVTQNVLYENQKQIANQFNKAISQIQESLTTTSTALGKLQDVVNQNAQ |
| | ALNTLVKQLSSNFGAISSVLNDILSRLDKVEAEVQIDRLITGRLQSLQTYVTQQL |
| | IRAAEIRASANLAATKMSECVLGQSKRVDFCGKGYHLMSFPQAAPHGVVFLH |
| | VTYVPSQERNFTTAPAICHEGKAYFPREGVFVFNGTSWFITQRNFFSPQIITTDN |
| | TFVSGNCDVVIGIINNTVYDPLQPELDSFKEELDKYFKN |
| | HTSPDVDLGDISGINASVVNIQKEIDRLNEVAKNLNESLIDLQELGKYEQYIKW |
| | PWYVWLGFIAGLIAIVMVTILLCCMTSCCSCLKGACSCGSCCKFDEDDSEPVL |
| | KGVKLHYT |

>sp|P59594|306-527 SARS-CoV-2 Spike protein receptor-binding domain

| | |
|---|---|
| SEQ ID | RVVPSGDVVRFPNITNLCPFGEVFNATKFPSVYAWERKKISNCVADYSVLYNS |
| NO: 3 | TFFSTFKCYGVSATKLNDLCFSNVYADSFVVKGDDVRQIAPGQTGVIADYNYK |
| | LPDDFMGCVLAWNTRNIDATSTGNYNYKYRYLRHGKLRPFERDISNVPFSPDG |
| | KPCTPPALNCYWPLNDYGFYTTTGIGYQPYRVVVLSFELLNAPATVCGPKLST |
| | DLIKNQCVNF |

>sp|P59594|424-494 SARS-CoV-2 Receptor-binding motif; binding to human ACE2

| | |
|---|---|
| SEQ ID | NTRNIDATSTGNYNYKYRYLRHGKLRPFERDISNVPFSPDGKPCTPPALNCYW |
| NO: 4 | PLNDYGFYTTTGIGYQPY |

1.-20. (canceled)

21. A method of measuring a presence, an absence, or a quantity of neutralizing antibodies against a coronavirus that functionally block binding between a spike protein of the coronavirus and Angiotensin-converting enzyme 2 (ACE2), the method comprising:
a) providing a biological sample obtained from a human subject that was administered a pharmaceutical formulation comprising a vaccine against the coronavirus, wherein the pharmaceutical formulation is formulated for human administration;
b) bringing the biological sample into contact with:
(i) a first peptide or protein comprising the ACE2 polypeptide or a portion thereof; and
(ii) a second peptide or protein comprising the spike protein or a portion thereof, wherein:
(1) the first peptide or protein and the second peptide or protein form a complex in an absence of neutralizing antibodies in the biological sample that block binding between the spike protein and ACE2 under conditions otherwise suitable for binding; and
(2) the first peptide or protein, the second peptide or protein, or a combination thereof comprises a detectable moiety;
c) detecting an absence, a presence, or a quantity of the complex formed in (b); and
d) measuring the presence, the absence, or the quantity of the neutralizing antibodies against the coronavirus, which is inversely correlated with the presence, the absence, or the quantity of the complex detected in (c), respectively.

22. The method of claim 21, wherein the biological sample is classified based on the measuring in (d) by a mobile application on a personal electronic device comprising a smartphone, a tablet, or a personal computer.

23. (canceled)

24. The method of claim 21, wherein the method does not consist of utilizing an immortalized cell or immortalized cell culture.

25. The method of claim 21, further comprising providing a result to a user on an electronic device regarding the presence, the absence, or the quantity of the neutralizing antibodies against the coronavirus in the biological sample.

26. The method of claim 21, further comprising:
(a) bringing the biological sample into contact with a capture moiety specific to one or more antibodies against the coronavirus; and
(b) detecting, in the biological sample, a presence, an absence, or a quantity of the one or more antibodies against the coronavirus, wherein the one or more antibodies against the coronavirus is not a neutralizing antibody.

27. The method of claim 26, wherein the one or more antibodies against the coronavirus belong to a class of antibodies comprising immunoglobulin A, immunoglobulin M, immunoglobulin G, or a combination thereof.

28. The method of claim 21, wherein the vaccine against the coronavirus is approved by a regulatory agency for prevention of a disease caused by the coronavirus.

29. The method of claim 21, further comprising administering a second dose of the vaccine against the coronavirus to the human subject.

30. (canceled)

31. (canceled)

32. (canceled)

33. (canceled)

34. (canceled)

35. (canceled)

36. (canceled)

37. A system for point of need or point of care comprising: a testing device or module to detect neutralizing antibodies against a coronavirus in a biological sample obtained from a human subject that was administered a pharmaceutical formulation comprising a vaccine against the coronavirus that is formulated for human administration, wherein the neutralizing antibodies functionally block binding between a spike protein of the coronavirus and Angiotensin-converting enzyme 2 (ACE2) to aid in the diagnosis of a disease or a condition caused by the coronavirus at the point of need or the point of care, the testing device comprising:
a) a first peptide or protein comprising the ACE2 polypeptide or a portion thereof; and
b) a second peptide or protein comprising the spike protein or a portion thereof, wherein the first peptide or protein and the second peptide or protein form a complex in an absence of neutralizing antibodies in the biological sample that block binding between the spike protein and ACE2 under conditions otherwise suitable for binding, and wherein the first peptide or protein, or the second peptide or protein comprises a detectable moiety.

38. The system of claim 37, further comprising a mobile application that runs on a personal electronic device, wherein the mobile application is programmed to generate a classification of a biological sample from the human subject as having a presence, an absence, or a quantity of one or more neutralizing antibodies against the coronavirus by analyzing image data from an image of the testing device at the point of need or the point of care to aid in the diagnosis of the disease or the condition caused by the coronavirus in the human subject.

39. The system of claim 37, wherein the testing device is portable.

40. The system of claim 37, wherein the system does not consist of a cell or a cell culture.

41. The method of claim 21, wherein the spike protein of the coronavirus comprises a receptor binding domain specific for binding with ACE2 in vivo.

42. The method of claim 21, wherein the coronavirus is a Severe acute respiratory syndrome-related (SARS) virus.

43. The method of claim 42, wherein the SARS is SARS-CoV-2.

44. The system of claim 37, wherein the spike protein of the coronavirus comprises a receptor binding domain specific for binding with ACE2 in vivo.

45. The system of claim 37, wherein the coronavirus is a Severe acute respiratory syndrome-related (SARS) virus.

46. The system of claim 45, wherein the SARS is SARS-CoV-2.

47. The system of claim 37, wherein the neutralizing antibodies against a coronavirus are detected using the testing device or module by contacting a biological sample with the first peptide or protein and the second peptide or protein, wherein a presence, an absence, or a quantity of neutralizing antibodies against a coronavirus in the biological sample are inversely correlated with a presence, an absence, or a quantity of a labeled complex formed between the first peptide or protein and the second peptide or protein, respectively, in the presence of the biological sample.

48. The system of claim 37, further comprising:
c) the biological sample contacted with the first peptide or protein and the second peptide or protein of the device or module, wherein the first peptide or protein and the second peptide or protein are bound to each other in the presence of the biological sample.

49. The method of claim 21, wherein the biological sample is obtained from one or more finger pricks of the human subject.

50. The system of claim 37, wherein the biological sample was obtained from one or more finger pricks of the human subject.


SEQUENCE LISTING (4 sequences)

SEQ ID NO: 1 - protein, 805 aa, Homo sapiens [sequence omitted]

SEQ ID NO: 2 - protein, 1255 aa, Severe acute respiratory syndrome-related coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 3 - protein, 222 aa, Severe acute respiratory syndrome-related coronavirus
RVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNF

SEQ ID NO: 4 - protein, 71 aa, Severe acute respiratory syndrome-related coronavirus
NTRNIDATSTGNYNYKYRYLRHGKLRPFERDISNVPFSPDGKPCTPPALNCYWPLNDYGFYTTTGIGYQPY